(19) United States
(12) Patent Application Publication
(10) Pub. No.: US 2025/0288274 A1
(43) Pub. Date: Sep. 18, 2025

(54) WEARABLE DEVICE AND OPERATION THEREOF

(71) Applicants: Andreas SCHUH, Mountain View, CA (US); Nelson L. JUMBE, Mountain View, CA (US); Peter REXELIUS, Mountain View, CA (US); Michael MORIMOTO, Mountain View, CA (US); Andrew URAZAKI, Mountain View, CA (US)

(72) Inventors: Andreas SCHUH, Mountain View, CA (US); Nelson L. JUMBE, Mountain View, CA (US); Peter REXELIUS, Mountain View, CA (US); Michael MORIMOTO, Mountain View, CA (US); Andrew URAZAKI, Mountain View, CA (US)

(21) Appl. No.: 18/862,299

(22) PCT Filed: May 3, 2023
(86) PCT No.: PCT/US2023/066539

§ 371 Date: Nov. 1, 2024

Related U.S. Application Data
(60) Provisional application No. 63/337,802, filed on May 3, 2022.

(51) Int. Cl. A61B 7/04; G16H 40/63; G16H 50/20

(52) U.S. Cl. CPC A61B 7/04; G16H 40/63; G16H 50/20; A61B 2560/0462; A61B 2562/0204

(57) ABSTRACT

A wearable device comprising: a vibroacoustic sensor directionally facing away from the wrist that can be placed against a body part of a user by movement and articulation of the arm and wrist. A wearable device comprising two or more vibroacoustic sensors which are positioned such that they can collect data from different auscultation points from a body of a user, when the wearable device is worn by the user. Methods of using the wearable devices

### TECHNICAL FIELD

This disclosure generally relates to wearable devices, such as but not limited to wearable devices including sensors that can measure signals associated with a user of the wearable device.

### BACKGROUND

Wearable devices for detecting physiological signals from a subject include fitness trackers, for example.

However, currently available fitness trackers are limited in their ability to measure, monitor, and diagnose health related issues. This is due to the limitation of the sensors integrated into the device. For example, with a photoplethysmographic sensor, the device is limited to subsurface vasculature of the wrist to detect oxygenation level of blood specifically at the wrist. With ECG electrodes and/or galvanic skin response electrodes, the signal obtained can only provide a heart rate and information on superficial analytes such as sweat. Other sensors including a thermometer, tonometer, and accelerometer only provide external information and are limited by artifacts caused by overall movement of extremities to provide accurate body data.

Many wrist wearable monitors can provide temperature and heart rate, but are unable to provide accurate blood pressure (i.e. systolic and diastolic blood pressure). This is predominantly due to the weaker signal at the wrist compared to other major arteries such as the carotid artery or femoral artery. Current technologies rely on the old-fashioned cuff-based system which requires a cuff and pressure application functionality which is cumbersome and uncomfortable on a wrist. Other technologies rely on pulse transit time, the time required for the arterial pressure wave to travel from the left ventricle to a distal arterial site such as the radial or ulnar artery. However, pulse transit time systems require calibration to an individual as changes in arterial compliance and blood density are inversely related factors. Furthermore, in peripheral arteries the arterial wall thickness and arterial radius is relatively small but a major factor in the pulse transit time calculation, thereby leading to variances in measured blood pressure. Other applications that rely on electrocardiogram photoplethysmography require the use to remain motionless for a 20 second to 1 minute duration and results are greatly affected by motion artifacts which would be difficult to factor out by programming alone. Additionally, pulse transit times calculated by ECG-based systems suffer from variability in the pre-ejection period that is independent from blood pressure values, resulting in decreased accuracy of blood pressure estimates. Although a combination of the above wearable sensors can be used to extrapolate one or more states of the user, it is far too difficult to identify and quantify the presence and progression of a disease due to the requirement of periodic calibration with approved gold standard technologies and many potential errors due to user errors caused by very narrow allowable use instructions.

It is therefore desired to provide a wearable device which overcomes or reduces the above-noted shortcomings,

### SUMMARY

The present technology relates to the development of wearable devices and their methods of use which address or overcome the above-noted shortcomings.

From a broad aspect, there is provided a wearable device including a vibroacoustic sensor. The wearable device may be configured to be worn on a wrist or an arm of a user and comprise a watch configuration, a wrist band, a bracelet, or the like. In other embodiments, the wearable device may be configured to be worn on an ankle or a leg of a user. In yet other embodiments, the wearable device may be configured to be worn on any other body part of the user.

In certain embodiments, the wearable device includes two or more sensors, one of which is the vibracoustic sensor. The secondary sensor may be one or more of: a second vibroacoustic sensor, a photoplethysmographic sensor, a bioelectric sensor, an oxygen saturation sensor, an ultrasonic sensor, an electrochemical sensor, and an environmental sensor.

In certain embodiments, the wearable device extends, at least partially, around a limb of the user when worn. The two or more sensors may be co-located in the same radial position. In other embodiments, the two or more sensors may be located in different radial positions.

In certain embodiments, one sensor may be positioned on an inner face of the wearable device and another sensor may be placed on an outer side of the wearable device.

In certain embodiments, the two or more sensors may be integrated in a single printed flexible circuit board. In embodiments in which the sensors are co-located in the same radial position, the flexible circuit board may be folded in a stacked fashion. In embodiments in which the sensors are located in different radial positions, the flexible circuit board may be embedded within the wearable device (e.g. wrist band).

In other embodiments, the two or more sensors are wirelessly connected.

The vibroacoustic sensor may comprise a voice coil type sensor which can detect frequencies in a range of about 0.1 Hz to about 160 kHz. The vibroacoustic sensor may have a diameter less than about 25 mm.

In certain embodiments, at least one of the two or more sensors comprise a foldable sensor device. The foldable sensor device may comprise a first substrate having a first sensor; a second substrate having a second sensor; and a first join member connecting the first substrate and the second substrate such that the first substrate and the second substrate are folded relative to each other to form a folded configuration having multiple substrate layers stacked relative to one another. Foldable sensors have been described in PCT/US2021/063151 filed Dec. 13, 2021, the contents of which are herein incorporated in their entirety.

In certain embodiments, the wearable device may be configured to provide the user with an indication of proper placement. For example, the wearable device may include a user interface, such as a screen, and the indication may comprise a visual signal on the screen. The visual signal may comprise a written message, a light, etc. In other examples, the wearable device may include a speaker, and the indication may comprise an audio signal from the speaker. In yet other examples, the wearable device may include a haptic component and the indication may comprise a haptic signal from the haptic component. In yet other embodiments, the indication may be provided in another manner, such as on another device.

The wearable device may include a positioning sensor to indicate adequate adherence of the sensor to the user and proper positioning. As a signal to the user of accurate positioning, the device may produce a distinct colored, steady light, when it is positioned properly on the body. In other embodiments, the indication to the user may comprise a blinking light with a first frequency (e.g. 1 s) when in an “on” mode, and a blinking light with a second frequency (e.g. 5 s) when in a “stand-by” mode. The “on” and “standby” modes may also be indicated by distinct colored lights. Audio commands may be used in conjunction or alternatively.

In certain embodiments, the wearable device includes a processor for executing a method. The method may include processing data collected by the at least two sensors.

In certain embodiments, the wearable device includes a memory for storing the data collected by the at least two sensors.

In certain embodiments, the processor of the wearable device is configured to transmit the collected data to another processor, such as that of a server, which processes the data.

From another broad aspect, there is provided a sensing system comprising the wearable device as described herein, and a processor of a computer system configured to process the data collected by the at least two sensors.

From a yet further aspect, there is provided a sensing system comprising: a plurality of the foldable sensor device and/or the wearable devices, as described herein, and in which each foldable sensor device is in the folded configuration; and a processor of a computing system, the processor being communicatively connectable to each of the foldable sensor devices. In certain embodiments, a first foldable sensor device of the plurality of foldable sensor devices comprises a first enclosure which is a wearable enclosure, and a second foldable sensor device of the plurality of foldable sensor devices comprises a second enclosure which is a wearable enclosure and is different than the first enclosure. For example, the first enclosure may be a watch and the second enclosure may be a patch, both the first and second foldable sensor devices configured to be worn by the same user on the user's wrist simultaneously but located in a different radial position. The radial position separation between the first enclosure and second enclosure is at least 1 degree off but ideally at least 5 degrees and no greater than 180 degrees.

From a further aspect, there is provided a method of processing data collected by the wearable device. The data may comprise vibroacoustic data. The method may be executed by a processor of the wearable device, another processor, or multiple processors. In this respect, the at least two sensors are configured to be communicatively connected to a processor of a computer system.

In certain embodiments, the method comprises simultaneously obtaining and time-syncing data from the at least two sensors.

In certain embodiments, the method is configured to determine one or more conditions of the user accurately through a machine learning algorithm that inputs data from the two sensors.

In certain aspects, the method is configured to monitor a health condition of a body using data collected by the at least two sensors of the wearable device.

In certain embodiments, the processor is configured to receive signal data from the first sensor and/or the second sensor, and to determine a condition of a body in contact with or in proximity to the foldable sensor device based on the signal data.

In certain embodiments, on detection of a given condition, the processor is configured to execute one or more of the following steps: (i) cause a generation of an alarm; (ii) communicate with another device.

In certain embodiments, the processor is configured to receive signal data from the first sensor and/or the second sensor, and to determine whether there is an activity of a body in contact with or in proximity to the foldable sensor device based on the received signal data.

In certain embodiments, the processor is configured to receive signal data from the first sensor and/or the second sensor, and to identify an activity of a body in contact with or in proximity to the foldable sensor device based on the received signal.

In certain embodiments, the processor is configured to trigger, based on a data collection protocol, one or both of the first sensor and the second sensor to one or more of: start collecting data, stop collecting data, start storing the collected data and stop storing the collected data.

In certain embodiments, the first sensor and the second sensor are connected to a power source and wherein the data collection protocol is based on a consideration of balancing battery life with collection of pertinent data or storage of pertinent data.

In certain embodiments, the data collection protocol is based on a predetermined time interval. In certain embodiments, the data collection protocol is based on a predetermined time interval and a trigger event.

In certain embodiments, the trigger event comprises one or more of an intensity of a detected activity, an intensity of a detected signal compared to a threshold intensity, and a frequency of a detected signal compared to a threshold frequency.

In certain embodiments, the wearable device and/or the system may be operated (assuming proper placement) on the skin, through clothes, over clothing and around obstacles.

According to certain embodiments, in the present technology, the devices, methods and systems may capture and process data relating to the subject such as one or more of:
Core body temperature of the subject
Heart audible/inaudible sounds of the subject
Lung audible/inaudible sounds of the subject
Gut audible/inaudible sounds of the subject
Central and peripheral vascular audible/inaudible sounds of the subject
Heart rate (plus variability) of the subject
Breath rate (plus variability) of the subject
Pulse and pulse delay & variability of the subject
Cough detection of the subject
Vocal changes of the subject
Stress and intensity of emotion state/emotional arousal changes of the subject via electrodermal activity (EDA)/galvanic skin response (GSR)
Physical stress/“readiness to train” score
Continuous or intermittent blood pressure monitoring of the subject
Continuous or intermittent ballistocardiography
Continuous or intermittent seismocardiography
Blood oxygen content of the subject.

The present technology has many uses including self-health screening/diagnosis, monitoring of disease progression, monitoring of reaction and effectiveness of a therapy, and contact tracing, etc., (which all have secure identity underpinnings).

In particular, the combination of continuous data collection and sensors capable of collecting “health-related” data allow the opportunity for the invention to catch certain diseases at their earliest stages. Some examples are provided below:

Valvular heart disease is present in around 2.5% of the United States population, with increased prevalence in older adults. (For context, atrial fibrillation is present in around 1%-2% of adults in the United States.) Many of these valvular diseases can be screened by a primary care physician using an acoustic stethoscope at an annual physical examination, but the prognosis of some acquired valvular heart diseases deteriorates with disease progression. For example, the average survival rate of symptomatic aortic stenosis without treatment is only two to three years. Early detection of heart murmurs at a more frequent cadence than an annual physical checkup may therefore benefit consumers.

Fully established heart murmurs are detectable with any acoustic or electronic sensor while auscultating the heart. However, the full spectrum/wide bandwidth vibroacoustics sensor design additionally allows heart murmurs to be detected at their earliest stages. This is because vibrations resulting from the transition from laminar (silent) to turbulent (noisy) fluid flow pass through the low frequency, infrasonic range, which is not detectable using traditional acoustic stethoscopes.

Contextualization of the vibroacoustic data additionally allows the opportunity to distinguish between physiologic and pathologic murmurs. For example, aerobically trained athletes may have morphological alterations to their hearts that may result in an innocent systolic ejection murmur. Framing the murmur in the context of heart rate, body composition, ECG signal, training recovery status, etc., may provide enough evidence to flag the murmur as potentially physiologic.

Respiratory sounds can be divided into normal and adventitious sounds, with the latter occurring during respiratory system diseases that affect air flow in the airways. Both normal and adventitious respiratory sounds are broad-spectrum and contain a wide range of frequencies, spanning the range from at least 100 Hz to 4000 Hz.

The broad frequency spectra for many respiratory sounds can be explained by air turbulence, harmonics, or a combination of both. Turbulence, random fluid motions within the flow field commonly associated with larger-volumetric flow rates, dissipates kinetic energy while cascading down time-and length-scales. The broad range of scales involved in this energy dissipation process results in a similarly broad range of frequencies produced by the turbulent flow. Normal respiratory sounds are often caused by turbulence, with a “noise”-like characteristic sound.

Harmonics, often associated with wheezes and rhonchi, arise from sinusoidal-like pressure fluctuations occurring at a “fundamental frequency” that produce integer multiples of the fundamental frequency. These combine to produce tones that are often associated with musical instruments. Wheeze fundamental frequencies are often within the range 100-1600 Hz, with typically up to three harmonic frequencies. Rhonchus fundamental frequencies often lie below 200 Hz.

The broad frequency spectra of respiratory sounds may include those frequencies below and above the frequency limits of human hearing, called infrasound and ultrasound, respectively. Infrasonic vibrations are often associated with turbulent flows; for example, though factors other than turbulence may be relevant, Korotkoff sounds are estimated to have more than 90% of their sound energy in frequencies below 25 Hz. More importantly, computational fluid dynamic studies have shown that infrasonic vibrations are also associated with the transition from a healthy state to a diseased state. Crackles, which are often characterized as short “explosive” sounds, may contain ultrasonic frequency components when not filtered through lung parenchyma or peripheral muscle/fat.

It is clear that the wide range of frequencies exhibited by respiratory sounds suggests that no particular frequency range holds absolute diagnostic powers over all others; rather, frequency band interactions and associations need to be considered when using respiratory sounds for diagnostic purposes. For example, COPD may be associated with wheezes, rhonchi, and coarse crackles. To help differentiate COPD from other pulmonary diseases, knowledge of the frequency bands associated with each of these sounds and their (possibly) simultaneous manifestations is critical in helping to make the COPD diagnosis.

The importance of frequency interactions holds true for various degrees of pulmonary damage resulting from pulmonary infection. Even for mild cases that do not produce adventitious respiratory sounds, subtle changes in normal breath sounds may occur throughout the wide frequency spectra associated with turbulent flow. More severe pulmonary infection cases that produce adventitious respiratory sounds, such as pneumonia-induced squawks and fine crackles, require examination of many different frequency bands due to the broad range of frequencies that may be produced during inspiration. In the most severe cases with acute respiratory distress syndrome (ARDS), adventitious sounds may still exist (e.g., fine crackles) and be superimposed with other wide-band vibrations, such as dyspnea or tachycardia. Animal studies of ARDS also suggest a shift in the ratio of high frequencies (400-600 Hz) to lower frequencies (150-300 Hz) as ARDS severity increases, as measured by PaO2/FiO2 ratio and static lung compliance, clearly demonstrating the potential need to consider frequency band interactions with disease diagnoses.

Analyses on pilot COVID-19 clinical trial data confirms the necessity of including broad frequency band interactions that allow classification of COVID-positive and-negative individuals. By using non-overlapping spectral bands, extracted from various segments of vibroacoustic data, to optimally search for a structured and transparent machine learning model to classify the COVID status of individuals, it was found that frequency band interactions are common in the features with highest predictive power. For example, it was observed that the frequency bands 16-30 Hz (containing infrasound) and 64-126 Hz interacted via a combination of time series addition and filtering in the same structured manner in more than one predictive feature.

The invention's ability to capture subtle differences in pulmonary health allow for applications in disease progression and, equally as important, monitoring of treatment/intervention effects. The latter is particularly important in home-based monitoring/follow-up care and in the communities where frequent diagnostic testing is not feasible from a logistical and cost perspective.

High blood pressure is commonly linked to brain, kidney, and heart diseases, and if left untreated, it can lead to stroke and coronary heart disease. Therefore, for outpatient care as well as general consumer health monitoring, there is great interest in being able to accurately and frequently measure blood pressure outside of a clinical setting using mobile or wearable devices. Many of these implementations rely on the relationship between pulse arrival time (PAT) and blood pressure, where PAT is measured as the time difference between the ECG R-wave and PPG peak during a heart cycle. Unfortunately, PAT is also affected by the heart's pre-ejection period (PEP), which may change independently of blood pressure changes.

There is some evidence that the S1 heart sound provides a better fiducial point for marking the opening of the aortic heart valve. The invention allows high-sensitivity collection of heart sounds to make this calculation feasible in a wearable form-factor. Moreover, contextualization of the data may help explain some of the variability that has prevented precise and stationary estimation of blood pressure in almost all attempts to date

Additionally, the invention's inclusion of “health-related” data allows for tracking of health- and wellness-related activities, including physical fitness enhancement. Monitoring health and performance tracking have been the one of the primary advantageous aspects of wearable technology. Not only does it help fitness enthusiasts by providing them with necessary health parameters, but it also aids patients by providing insights on their conditions. As a result of the COVID-19 pandemic, the general public, employers, and insurers are becoming more health conscious, and this has led to an increase in shipment of fitness trackers and smartwatches. The sensors in the invention can be programmed to optimize workout sessions based on previous week sleep and stress targets, for example. This is known as the measurement of an athlete's “readiness to train.” Training should be planned in a progressive manner in order to optimize potential adaptations. The Plisk and Stone fitness-fatigue paradigm illustrates that there are two after-effects of training: fitness and fatigue. In order to optimize an athlete's preparedness for performance and training adaptations, the effects of fatigue should be minimized over time. Excessive and poorly planned training loads can ultimately lead to non-functional overreaching and overtraining syndrome. Monitoring an athlete prior to a training session to assess how ready they are for the session is often done in the applied setting to assess their levels of fatigue and potential to optimize the adaptations that can be realized from the planned session. Level 42 AI has proof-of-concept data demonstrating the utility of vibroacoustic data in predicting a readiness score. Augmenting existing HR/HRV-based algorithms with this contextualized sensor data may provide a more accurate estimate of a patient's functional training status. In this case, long periods of data collection would no longer be required to estimate a readiness score, providing an advantage over offerings by other fitness-focused wearable products.

Digital identity management is fundamental for the further development of the Internet Economy. The management of digital identity enables trusted remote interactions between an organization and an individual. Managing the digital identity lifecycle generally involves several processes:
1 In order to be known by the system, the individual must first register with it and the conditions related to his/her identity or identity attributes must be checked so he/she can be provided with a set of credentials; this is the so-called registration or enrollment process.
2. Appropriate permissions and privileges to access the organization's resources must be assigned to the individual, a process often called authorization.
3 To access resources, the individual makes an identity claim that can be verified: he/she logs into the system with the credentials provided during the registration process. This authentication process establishes confidence in the user's identity.
4. The result of the authentication process is used in a process called access control, whereby the system checks that the individual has the appropriate authorization to access the resource.
5. When the individual is not associated anymore with the system, a revocation process must take place whereby his/her credentials are rescinded.

Digital identity management is essential to the security of the organisation that grants access to resources in its information system. It is also essential to the security of the individual who accesses these resources, particularly when they belong or relate to him/her (e.g. money in a bank, or personal data such as a medical record). By offering security and privacy, digital identity management enables the establishment of a trusted relation-ship between remote parties.

Digital identity management does not offer a binary choice between full assurance or no assurance regarding the parties to an interaction. It offers a range of levels of assurance, as appropriate (e.g. low, medium or high). The rationale for selecting the level of assurance primarily includes its alignment with the level of risk carried by the interactions between the parties. If the level of assurance is lower than the level of risk, the parties are likely not to interact (e.g. a low level of assurance will not enable to secure a high value transaction). Reversely, asking individuals to provide too high a level of assurance might deter them from carrying out medium or low risk inter-actions, which do not seem to demand it. Indeed, in the physical world, we are used to being asked to prove our identity or to exhibit identity attributes when it is justified by the level of risk involved in a given interaction. Ensuring proportionality is even more important online because of the capacity of information systems to store identity information and transaction records indefinitely.

Furthermore, in some cases, the delivery of services online enables a higher degree of privacy protection than what is possible offline. For example, it is difficult in the physical world to validate identity attributes like age or marital status without identifying an individual or to establish legally binding trusted offline interactions based on the use of pseudonyms. Such privacy protective mechanisms are however possible online.

Ensuring the highest level of privacy protection that technology enables, consistent with the appropriate level of assurance, is critical to further developing the market for online services, and in particular medium and high value ones.

In certain embodiments, the present technology can be used to generate a new system of verification mode through which a person's identity is validated by comparing captured biometric data with ready-made template. The generated, unique Decentralized Identifier technology (uDIDt) defined in this disclosure is a new type of globally unique identifier designed to enable individuals and organizations to generate their own identifiers (uDIDt-VeCx) using systems they trust, and to prove control of those identifiers (authenticate) using cryptographic proofs (eg., digital signatures, privacy-preserving biometric protocols, etc).

As personal identification and biosensor data becomes more relevant, methods and systems for cryptographically encoding specific combinations and subsets of biometric, biosignature, or biofield data to furnish distributed, unforgeable, and selectively non-deanonymizable personal or ecological identifiers becomes more important.

Biosensors are considered as devices that transform biophysiological information into an analytically useful signal. For accurate and timely biometric evaluation, the biosensors need (i) accurate measurement, (ii) rapid assessment, and (iii) selective detection. In certain embodiments, biometric data is collected and/or monitored in one or both of a baseline phase and a base-line update phase along with environmental (contextual) determinants of physiological and pathophysiological health conditions to correct for signal drift).

In the baseline phase, biometric data may be collected and/or monitored over 1 to 5 days, 1 to 4 days, 1 to 3 days, 1 to 2 days, 2 to 5 days, 3 to 5 days, 4 to 5 days, 1 to 3 days, 2 to 3 days. Data collection may be continuous or in data segments.

In the update phase, biometric data may be collected and/or monitored for 1 to 25 seconds, 5 to 25 seconds, 10 to 25 seconds, 15 to 25 seconds, 20 to 25 seconds, 1 to 20 seconds, 1 to 15 seconds, 1 to 10 seconds, 5 to 10 seconds, 5 to 15 seconds.

In certain embodiments, methods and systems of creating the uDIDt-VeCx comprises acquiring data segments of about 15 s to about 20 s in length, or about 10 to about 25 s, or any other data segment length which satisfies data quality and data quantity requirements.

In certain embodiments, credentialing for health/travel passports during pandemic or disaster emergencies requires collection of data from about 2 days to about 4 days for continuous health characterization and baselining.

In certain embodiments, updates using baselined data requires a shorter confirmatory data read or top-up from about 5 seconds to about 10 seconds.

In certain embodiments, the method comprises acquiring biometric data of a subject at a first point in time, and storing in a database (“pre-screening step). The stored data may be used to generate the identifier at the first point in time, or at a second point in time which is later than the first point in time. The method further comprises, at the second point in time, using the stored data or the identifier as a “verifiable credential” or “keycard” for entry.

In certain embodiments, the method may further comprises obtaining digital image data of the subject's face or other body parts for ID purposes. This may occur at the first or second points in time.

The pre-screening process may be carried out over a period of about 1 to 5 days.

In certain embodiments, the generated unique identifier is used as a health passport. In certain embodiments, the baseline data is ephemeral (can be deleted, over written, or loses validity).

In certain embodiments, the generated unique identifier can be used for contact tracing. This can be helpful for maintaining privacy.

Furthermore, certain aspects and embodiments of the present technology may include for example multi-dimension data stream fusion, with edge and cloud-based domain adaptable recursive stochastic synthesis programming. Certain embodiments of the present technology also provide for the use of said sensor devices with communications networks and computing platforms, to create models, detect changes, and perform detailed condition-based monitoring and health failure prediction.

By monitoring signal data relating to the subject and the environment, correlations can be made between a condition of the subject and the environment, and such correlations can be used to enhance a current condition of the subject (whether living or inanimate), maintain a current condition of the subject, prevent a certain condition (such as disease states) of the subject, and/or predict certain conditions of the subject. For example, such data may allow anomalous health incidents (AHI) to be identified. One example of an AHI is due to directed energy weapons such as those associated with the Havana Syndrome. Directed energy weapons may include beams of energies over a broad spectrum of infrasound, radio, sonic and microwave frequencies that cause a range of temporary or permanent health effects which in certain cases resemble decompression sickness. In certain embodiments, the present technology can therefore establish a baseline for a subject by monitoring physiological parameters such as respiration, blood pressure, electrocardiogram signal, sleep analysis. The baseline may be a dynamic baseline, i.e. updated. An AHI may be identified as anomalous perturbations to the baseline.

Therefore, the present technology aims to provide solutions towards health state/condition, health status condition monitoring, symptom screening and diagnostics, as well as overall physical performance enhancement in living bodies. This differs from traditional approaches of current symptom and disease management.

This objective is also important in health monitoring of inanimate structures, such as buildings, dams, bridges, as well as in the efficient use of animate structures such as rotating and reciprocating machinery, vibrating structures, mechanical facilities and technical systems in the industrial and construction sector, and wherever else machines are used. Humans, rotating structures and machines have traditionally been placed on an arbitrary service plan; milestone-driven vaccine and/or yearly physical physician office visits, or mileage/rotation triggered maintenance, where late maintenance means destructive wear and a risk of production downtime.

According to certain aspects, there are provided devices, methods and systems for condition-based monitoring and/or health failure prediction in animals, plants, as well as non-living structures.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 illustrates a system for implementing and/or executing any of the devices and/or methods described herein in accordance with various embodiments of the present technology;

FIG. 2 illustrates an example computing environment in accordance with various embodiments of the present technology;

FIG. 3 illustrates a perspective view of a wearable device in accordance with various embodiments of the present technology;

FIG. 4 illustrates a side view of the wearable device in accordance with various embodiments of the present technology;

FIG. 5 illustrates an end view of the wearable device in accordance with various embodiments of the present technology;

FIG. 6 illustrates a top view of the wearable device in accordance with various embodiments of the present technology;

FIG. 7 illustrates a wearable device with a vibroacoustic sensor on its band in accordance with various embodiments of the present technology;

FIGS. 8, 9, 10, and 11 illustrate other embodiments of wearable devices in accordance with various embodiments of the present technology;

FIGS. 12A, 12B, and 12C illustrate wearable devices with modular displays and bands in accordance with various embodiments of the present technology;

FIG. 13 illustrates a wearable device positioned on an individual's gut in accordance with various embodiments of the present technology;

FIG. 14 illustrates a wearable device positioned renally in accordance with various embodiments of the present technology;

FIG. 15 illustrates a wearable device positioned on an individual's gut in accordance with various embodiments of the present technology;

FIG. 16 illustrates a wearable device positioned on an individual's lung in accordance with various embodiments of the present technology;

FIG. 17 illustrates a wearable device positioned on an individual's lung in accordance with various embodiments of the present technology; and

FIG. 18 illustrates a wearable device positioned on an individual's carotid artery in accordance with various embodiments of the present technology.

It should be noted that, unless otherwise explicitly specified herein, the drawings are not to scale.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified embodiments of the present technology. As persons skilled in the art would understand, various embodiments of the present technology may be of greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and embodiments of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry and/or illustrative systems embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Some or all of the functions described herein may be performed by a cloud-based system. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown. Moreover, it should be understood that one or more modules may include for example, but without being limitative, computer program logic, computer program instructions, software, stack, firmware, hardware circuitry, or a combination thereof.

### Systems

### Computing Environment

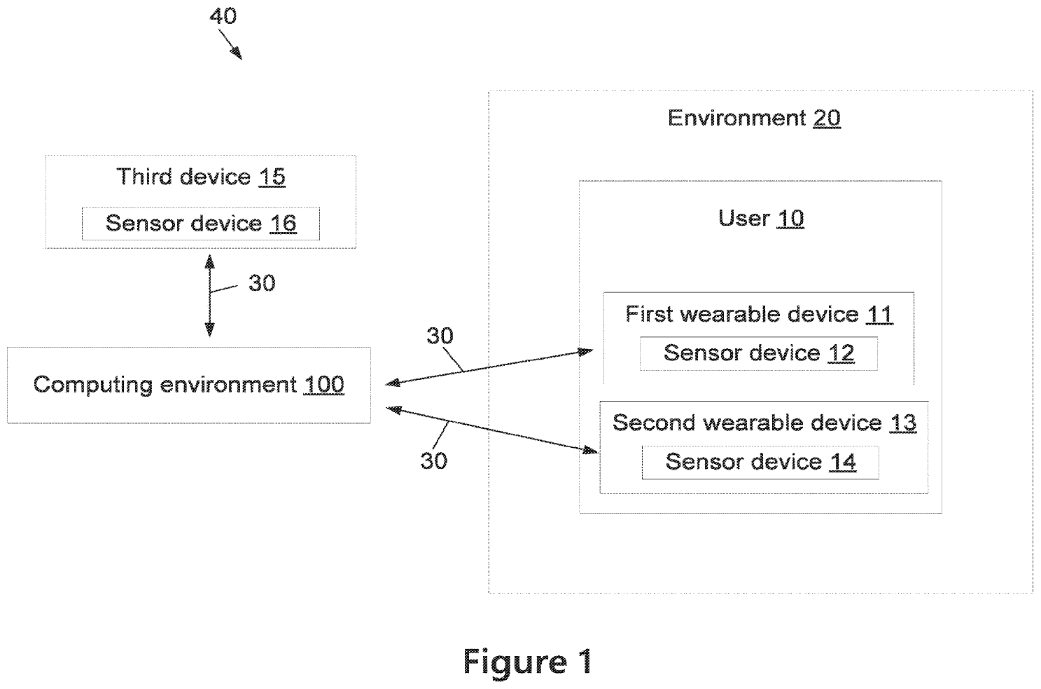

FIG. 1 illustrates a system 40 for implementing and/or executing any of the devices and/or methods described herein such as for example monitoring, enhancing, preventing or predicting a condition of a user 10 in an environment 20. In certain embodiments, the system comprises a first wearable device 11 including two or more sensors in a sensor device 12. Optionally, there is provided a second wearable device 13 including one or more sensors in a sensor device 14, and/or a third device 15 including one or more sensors in a sensor device 16. The first wearable 11, and optionally the second wearable device 13, and/or third device 15 are communicatively coupled to a processor 110 of a computing environment 100 via a network 30.

The first wearable device 11 may be worn by the user 10 and may be a watch. The sensor device 12 may record data about the user 10 and/or the environment 20 surrounding the user 10.

The two or more sensors of the first wearable device 11 may simultaneously record data about the user 10 and/or environment 20. Timestamped data may be collected from each of the at least two sensors.

The computing environment 100 may be a standalone device (as illustrated) and/or integrated within the first wearable device 11. The computing environment 100 may be integrated in an intelligence coordinator device (e.g. a microcontroller). The intelligence coordinator device may gather data from multiple sensor devices and/or other devices. Individual devices may send alerts to the intelligence coordinator device, such as after detecting an anomalous event.

In yet other embodiments (not shown), there may be provided additional wearable devices or other devices with sensors communicatively coupled to the processor 110 of the computing environment 100 via the network 30.

The environment 20 may include the user 10 and/or other users (not illustrated). Data about the other users and/or about the environment may be collected, such as by wearable devices being worn by the other users. Data collected about the other users in the environment 20 may be collected by the computing environment 100 and processed as environmental data corresponding to the user 10. In other words, the data collected from the other users in the environment 20 may be used as data describing the environment 20.

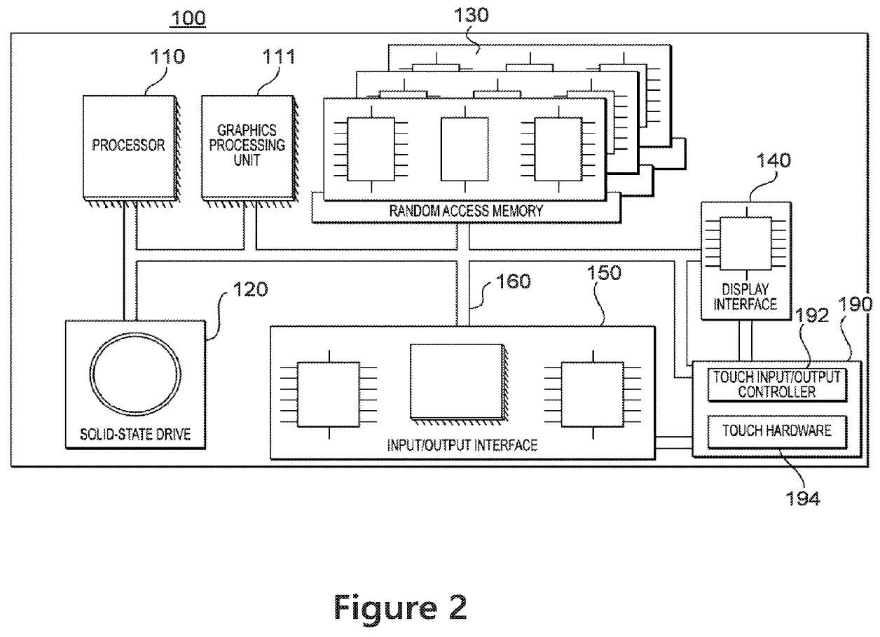

FIG. 2 illustrates an embodiment of the computing environment 100. In some embodiments, the computing environment 100 may be implemented by any of a conventional personal computer, a network device and/or an electronic device (such as, but not limited to, a mobile device, a tablet device, a server, a controller unit, a control device, etc.), and/or any combination thereof appropriate to the relevant task at hand. In some embodiments, the computing environment 100 comprises various hardware components including one or more single or multi-core processors collectively represented by processor 110, a solid-state drive 120, a random access memory 130, and an input/output interface 150. The computing environment 100 may be a computer specifically designed to operate a machine learning algorithm (MLA). The computing environment 100 may be a generic computer system.

In some embodiments, the computing environment 100 may also be a subsystem of one of the above-listed systems. In some other embodiments, the computing environment 100 may be an “off-the-shelf” generic computer system. In some embodiments, the computing environment 100 may also be distributed amongst multiple systems. The computing environment 100 may also be specifically dedicated to the implementation of the present technology. As a person in the art of the present technology may appreciate, multiple variations as to how the computing environment 100 is implemented may be envisioned without departing from the scope of the present technology.

Those skilled in the art will appreciate that processor 110 is generally representative of a processing capability. In some embodiments, in place of or in addition to one or more conventional Central Processing Units (CPUs), one or more specialized processing cores may be provided. For example, one or more Graphic Processing Units 111 (GPUs), Tensor Processing Units (TPUs), and/or other so-called accelerated processors (or processing accelerators) may be provided in addition to or in place of one or more CPUs.

System memory will typically include random access memory 130, but is more generally intended to encompass any type of non-transitory system memory such as static random access memory (SRAM), dynamic random access memory (DRAM), synchronous DRAM (SDRAM), read-only memory (ROM), or a combination thereof. Solid-state drive 120 is shown as an example of a mass storage device, but more generally such mass storage may comprise any type of non-transitory storage device configured to store data, programs, and other information, and to make the data, programs, and other information accessible via a system bus 160. For example, mass storage may comprise one or more of a solid state drive, hard disk drive, a magnetic disk drive, and/or an optical disk drive.

Communication between the various components of the computing environment 100 may be enabled by a system bus 160 comprising one or more internal and/or external buses (e.g., a PCI bus, universal serial bus, IEEE 1394 “Firewire” bus, SCSI bus, Serial-ATA bus, ARINC bus, etc.), to which the various hardware components are electronically coupled.

The input/output interface 150 may allow enabling networking capabilities such as wired or wireless access. As an example, the input/output interface 150 may comprise a networking interface such as, but not limited to, a network port, a network socket, a network interface controller and the like. Multiple examples of how the networking interface may be implemented will become apparent to the person skilled in the art of the present technology. For example the networking interface may implement specific physical layer and data link layer standards such as Ethernet, Fibre Channel, Wi-Fi, Token Ring or Serial communication protocols. The specific physical layer and the data link layer may provide a base for a full network protocol stack, allowing communication among small groups of computers on the same local area network (LAN) and large-scale network communications through routable protocols, such as Internet Protocol (IP).

The input/output interface 150 may be coupled to a touchscreen 190 and/or to the system bus 160. The touchscreen 190 may be part of the display. In some embodiments, the touchscreen 190 is the display. The touchscreen 190 may equally be referred to as a screen 190. In the embodiments illustrated in FIG. 1B, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the system bus 160. The display interface 140 may include and/or be in communication with any type and/or number of displays. In some embodiments, the input/output interface 150 may be connected to a keyboard (not shown), a mouse (not shown) or a trackpad (not shown) allowing the user to interact with the computing device 100 in addition to or instead of the touchscreen 190.

According to some embodiments of the present technology, the solid-state drive 120 stores program instructions suitable for being loaded into the random access memory 130 and executed by the processor 110 for executing acts of one or more methods described herein. For example, at least some of the program instructions may be part of a library or an application. Some or all of the components of the computing environment 100 may be integrated in a multi-layer sensor device and/or in communication with the multi-layer sensor device. The processor may be configured to process the data obtained by the multi-layer sensor device, and provide an output, such as to a smartphone of an operator of the system.

### Sensors

Present systems and devices, in certain embodiments, include sensors for capturing, and optionally processing, data associated with a user or of an environment of the user.

The one or more sensors used in the present technology is not particularly limited. The one or more sensors used in the present technology may include sensor stacks and/or other plug-and-play devices and/or systems.

In certain embodiments, the one or more sensors comprise a sensor array. The sensors may be selected from:

Passive and/or active vibrometry and vibroacoustic sensors. In certain embodiments, the sensor is a vibroacoustic sensor for detecting vibroacoustic signals. In some embodiments, transmission of vibroacoustic waves may occur through an intermediate medium such as air.

In some embodiments, the vibroacoustic sensor may have a bandwidth suitable for detecting vibroacoustic signals in the infrasound range, such as a bandwidth ranging from about 0.01 Hz to at least about 20 Hz. Furthermore, in some embodiments, the vibroacoustic sensor may have wider bandwidths covering a wider spectrum of infrasound-to-ultrasound, such as a bandwidth ranging from about 0.01 Hz to at least 160 kHz. In some embodiments, the biological vibroacoustic signal component extracted from the detected vibroacoustic signal may have a bandwidth ranging from about 0.01 Hz to 0.1 Hz.

For example, in some embodiments the vibroacoustic sensor may have an overall bandwidth ranging from about 0.01 Hz to at least about 50 kHz, from about 0.01 Hz to at least about 60kHz, from about 0.01 Hz to at least about 70 kHz, from about 0.01 Hz to at least about 80 kHz, from about 0.01 Hz to at least about 90 kHz, from about 0.01 Hz to at least about 100 kHz, from about 0.01Hz to at least about 110 kHz, from about 0.01 Hz to at least about 120 kHz, from about 0.01 Hz to at least about 130 kHz, from about 0.01 Hz to at least about 140 kHz, from about 0.01 Hz to at least about 150kHz, from about 0.01 Hz to at least about 160 kHz, from about 0.01 Hz to more than about 150 kHz.

The sensor may, in some embodiments, comprise a single sensor that provides one or more of the abovementioned bandwidths of detected vibroacoustic signals.

In some other embodiments, the sensor may include a suite or array of multiple sensors, each having a respective bandwidth range forming a segment of the overall vibroacoustic sensor bandwidth. At least some of these multiple sensors may have respective bandwidths that at least partially overlap in certain embodiments. In other embodiments, the multiple sensors do not have overlapping bandwidth ranges. Accordingly, various sensor bandwidths may be achieved based on a selection of particular sensors that collectively contribute to a particular vibroacoustic sensor bandwidth. In other words, bandwidth extension and linearization approach (bandwidth predistortion) may utilize modular sensor fusion and response feedback information, such as to compensate for bandwidth limitations of any particular single sensor with overlapped combinations of sensors to cover a wider bandwidth with optimal performance.

Such sensors may be used for detecting chronic pulmonary disease, lung consolidation, diffuse alveolar damage, vascular injury, and/or fibrosis, pulmonary embolism, and disseminated intravascular coagulation (DIC) cardiac structural disorders such as left ventricular hypertrophy, carotid disease, coronary disease, to name a few.

Certain example sensors for passive and/or active vibroacoustic sensors are described in PCT/US2021/046566 filed Aug. 18, 2021, the contents of which are herein incorporated by reference. In certain embodiments, passive vibroacoustic sensors are used which can detect frequencies of about 0.1 Hz to about 160 kHz. In certain embodiments, active vibroacoustic sensors are used which can detect frequencies of about 10 Hz to about 10 GHz.

More specifically, in certain embodiments, one or more of the sensors comprises a vibroacoustic transducer of a voice coil type. The voice coil transducer comprises a magnet housing having a cylindrical body portion with a bore and a flange extending radially outwardly from the cylindrical body portion. An iron core such as soft iron or other magnetic material is attached to the cylindrical body portion and lines the bore of the cylindrical body portion. The iron core extends around the bore of the cylindrical body portion as well as across an end of the cylindrical body portion. The iron core has an open end. A magnet is positioned in the bore and is surrounded by, and spaced from, the iron core to define a magnet gap. A voice coil, comprising one or more layers of wire windings supported by a coil holder, is suspended and centered in relation to the magnet gap by one or more spiders. The wire windings may be made of a conductive material such as copper or aluminum. A periphery of the spider is attached to the frame, and a center portion is attached to the voice coil. The voice coil at least partially extends into the magnet gap through the open end of the iron core. The one or more spiders allow for relative movement between the voice coil and the magnet whilst minimizing or avoiding torsion and in-plane movements. The voice coil transducer is attached to a diaphragm.

Additionally, the voice coil transducer is attached to the frame by the support members. Rotational movement of the frame relative to the frame is limited. Movements induced in the acoustic waves will cause the diaphragm to move, in turn inducing movement of the voice coil within the magnet gap, resulting in an induced electrical signal.

In certain variations of the voice coil transducer, the configuration of the transducer is arranged to pick up more orthogonal signals than in-plane signals, thereby improving sensitivity. For example, the one or more spiders are designed to have out-of-plane compliance and be stiff in-plane. The same is true of the diaphragm whose material and stiffness properties can be selected to improve out-of-plane compliance. The diaphragm may have a convex configuration (e.g. dome shaped) to further help in rejecting non-orthogonal signals by deflecting them away. Furthermore, signal processing may further derive any non-orthogonal signals e.g. by using a 3 axis accelerometer. This either to further reject non-orthogonal signals or even to particularly allow non-orthogonal signals through the sensor to derive the angle of origin of the incoming acoustic wave.

It will be appreciated that different uses and different sizes of the sensing device may require different sensitivities and face different noise/signal ratios challenges. For example, higher sensitivity and increased signal/noise ratio will be required for clothing contact uses compared to direct skin contact uses. Similarly, higher sensitivity and increased signal/noise ratio will be required for non-contact uses compared to contact uses. Therefore, in order to provide sensing devices having sensitivities and signal/noise ratios suitable for different form factors (e.g. contact or non-contact uses), developers have discovered that modulation of certain variables can optimize the voice coil transducer for the specific intended use: magnet strength, magnet volume, voice coil height, wire thickness, number of windings, number of winding layers, winding material (e.g. copper vs aluminum), and spider configuration. Developers also discovered that adaptation of the configuration of the spider contributed to increasing sensitivity and signal/noise ratio increases. More specifically, it was determined via experiment and simulation that making the spider more compliant such as by incorporating apertures in the spider, increased sensitivity. Apertures also allow for free air flow. In certain embodiment, the sensor comprises a voice coil transducer with the properties listed below

| Parameter | Present technology 1 | | Present technology 2 | |
|---|---|---|---|---|
| Impedance | 150 ohms ± 2% | | 150 ohms ± 2% | |
| DC Resistance (Re) | 150 ohms ± 2% | | 150 ohms ± 2% | |
| Voice Coil Inductance (Le) | 7.5 mH at 1 kHz / | | 8.46 mH at 1 kHz / | |
| | 2.5 mH at 10 kHz | | 2.7 mH at 10 kHz | |
| Coil Resonant Frequency | 80-170 | Hz | 90 Hz ± 2% | |
| (Fs) | | | | |
| Total Q (Qts) Inverse of | 0.25 to 0.65 | | 0.85-0.90 | |
| damping | (depending on exciter) | | | |
| | 100 mg to 100 g | | | |
| | depending on no. | | | |
| | windings) | | | |
| Moving Mass (Mms) | 100 mg to 100 g | | 1.15 | g |
| | (depends on number of | | | |
| | windings) | | | |
| | For test exciters | | | |
| | specifically 1.15 g | | | |
| Mechanical Compliance of | 0.4 to 3.2 | mm/N | 3.2 | mm/N |
| Suspension (Cms) (inverse | | | | |
| of suspension) | | | | |
| BL Product (BL) | 18.5 N/Amp (same | | 18.5 | Tm |
| | as Tm) | | | |
| Voice Coil Diameter | 25 | mm | 25 | mm |
| RMS Power Handling | 2 | W | 2 | W |
| Wire Diameter | 0.05 | mm | 0.05 | mm |
| Number of windings | 208 | | 208 | |
| Number of Layers | 4 | | 4 | |
| Magnet Size | 24 mm × 3.5 mm | | 24 mm × 3.5 mm | |
| Overall Outside Diameter | 50.5 mm (5 × 5 × 0.1 to | | 60 mm and 65 mm (oval | |
| | 50 × 50 × 10) | | shaped) | |
| Overall Depth | 20.5 | mm | 27 | mm |
| Inductance / moving mass | at least 6.52 mH per | | 7.36 mH per gram at 1 kHz | |
| ratio | gram at 1 kHz | | | |
| Mechanical compliance/ | at least 0.348 mm/N per | | 2.78 mm/N per gram | |
| moving mass ratio | gram | | | |
| BL product / moving mass | at least 16 N/Amp per | | 16.09 N/Amp per gram | |
| ratio | gram | | | |
| (BL x mechanical compliance)/ | 51.48 [T*m\hat{ }2 / (N * g)] | | 51.48 [T*m\hat{ }2 / (N * g)] | |
| moving mass | | | | |
| Wright Parameters | | | | |

| | | |
|---|---|---|
| K(r) | 26-27 | 23 |
| X(r) | 0.175-0.185 | 0.194 |
| K(i) | 0.00709-0.01118 | 0.032 |
| X(i) | 0.827-0.866 | 0.739 |

Bio-electric sensors, such as multi-lead ECG. Such sensors may be used for detecting conduction block, ventricular tachycardia, and/or ventricular fibrillation, cardiovascular disease, hypertension, diabetes. The most common complications include arrhythmia (atrial fibrillation, ventricular tachyarrhythmia, and ventricular fibrillation), cardiac injury, heart failure. QT prolongation, etc., for example, Changes in the QT interval in general-care patients who are undergoing treatment with drugs that can prolong QT intervals and may cause life threatening arrhythmias. On certain embodiments, widefield capacitive electric potential sensors are used. The bio-electric sensors may be able to record measurements with and/or without physical or resistive coupling. The bio-electric sensors may be configured to obtain Electrocardiogram (ECG), Electroencephalogram (EEG), Electrooculograph (EOG), and/or Electromyograph (EMG) readings. The bio-electric sensors may be used for imaging, security, and human-machine interfaces and devices that can “see” through walls.
Bio-electric sensors such as sensors for detecting Galvanic Skin Response (GSR), also referred to as Electrodermal Activity (EDA)/Electrodermal Response (EDR) or Skin Conductance (SC), and Psychogalvanic Reflex (PGR)—rapidly changing physiological characteristics of the largest organ of the human body—the skin. The skin functions as the principal interface between organism and environment. Together with other organs, it is responsible for bodily processes such as the immune system, thermo-regulation, and sensory-motor exploration.
Bio-electric sensors such as a non-contact electroencephalography (EEG) device, can detect a state of a person in proximity to the non-contacting sensor. The detected state may include an emotional state, a cognitive load state, and/or an alertness state of the person in proximity to the non-contacting sensor. When a processor in communication with and/or integrated within the non-contact device detects a pattern corresponding to a predetermined state of the person in proximity to the non-contacting sensor, the processor can transmit an action signal to another device to take a subsequent action, such as displaying an alert and/or or generating a “smart-alarm”.
Oxygen saturation sensors, such as Red+Green LED-O₂ saturation or Red LED plus IR LED-Normal oxygen levels are at least 95%. Some patients with chronic lung disease or sleep apnea can have normal levels around 90%. Oxygen saturation is a crucial measure of how well the lungs are working. Oxygen saturation measured by pulse oximetry is most frequently used to monitor patients with lung and heart disorders, who are at risk of low levels of blood oxygen. Oxygen saturation is also used to monitor patients before, during, and after surgery, including during anesthesia; to monitor patients on certain medications that may reduce respiration and lung function; to assess the lung function of people with conditions that can cause reduction of blood oxygen levels, including COPD, asthma, acute respiratory distress syndrome (ARDS), anemia, pneumonia, lung cancer, cardiac arrest, and heart failure, among others; to assess individuals with sleep disorders such as sleep apnea.
Aroma, gas and smell sensors. These e-nose solutions can include optical, surface acoustic wave (SAW), electrochemical, capacitive, catalytic, semiconductor gas sensors, electrical variation methods, imprinted polymer sensors, etc. rely on the following substances as a sensing.

Ultrasonic based acoustic sensors are principally classified as (1) ultrasonic, (2) attenuation, and (3) acoustic impedance. The major method for detection of sound velocity is to determine the time-of-flight that measures the travel time of ultrasonic waves at a known distance to calculate their speed of propagation. The measured gas speed is used for (1) identification of gases by determining gas properties such as gas concentration, which is related to the difference of sound propagation time, and for (2) determining the components or the molar weight of various gases in mixtures proceeding from thermodynamic considerations. Generally, ultrasonic sensors can overcome some shortcomings of gas sensors such as short lifetime.

Ecological, atmospheric, and environmental infrasound-to-terahertz sensors may be included in a broad-based, multi-focal, and/or adaptable technology solution. These sensors may be used to detect, identify, and/or defend against infrasound-to-ultrasound, electromagnetic frequency (EM), radio frequency (RF), laser, and/or magnetic resonance yoked instruments.

Attenuation is the energy loss due to thermal losses and scattering when an acoustic wave propagates through a medium. Each gas demonstrates particular attenuation, therefore providing means to determine target gases. Gas attenuation can be utilized together with sound velocity to determine gas properties. However, the attenuation method is not so reliable as the method of sound speed because it is prone to the presence of particles and droplets or the turbulence in the gas.

Acoustic impedance is typically employed for assessment of gas density. Therefore, by the quantified acoustic impedance and speed of sound, the density of a gas could be found out. In any case, the quantification of the acoustic impedance of gases is remarkably troublesome, particularly in a process environment and consequently it is rarely used in practice.

Semiconductor gas sensing depends on the variation of the oxide on the surface of a metal-oxide-semiconductor (MOS) stack which is transformed into a change of the sensor's electrical resistance as a means to detect gases via redox reactions between the target gas(es) and the oxide surface. To obviate the high operating temperature requirement, which can limit their application, microheaters produced by VLSI CMOS technology are used. Another issue, the relatively lengthy time needed for the gas sensor to recover after each gas exposure, which is impractical for applications where gas concentration changes quickly, is overcome by using nanodimension structures (e.g., nanowires and nanotubes).

Polymer-based sensors detect aromas and gases released by the body using a polymer layer that is changes its physical properties (mass, dielectric properties) upon gas absorption. Polymer sensors detect volatile organic compounds such as alcohols, formaldehyde, aromatic compounds or halogenated compounds released by skin and breath. Polymer gas sensors possess benefits such as high sensitivities and short response times. Their shortcomings include lack of long-term stability, reversibility and reduced selectivity.

Carbon nanotube sensors overcome the problem of insufficient sensitivity at room temperature observed by MOS sensors. The properties of carbon nanotubes (CNTs) allow the development of high-sensitive gas sensors. CNT sensors demonstrate ppm levels response for a range of gases at room temperature, which makes them perfect for low power applications. Their electrical properties carry high sensitivity to very small quantities of gases such as carbon dioxide, nitrogen, ammonia, oxide, and alcohol at room temperature (unlike MOS sensors, which should be heated by a supplementary heater in order to operate normally). Multiwall CNTs have been employed for remote sensing of carbon dioxide (CO2), ammonia (NH3), and oxygen (O2). To enhance selectivity and sensitivity of sensing, CNTs are often combined with other materials. Moisture absorbing materials detect moisture, because their dielectric constant can be tuned to be altered by the water content in the environment. They can be used also as a substrate of the devices of the present technology because the dielectric constant of moisture absorbing materials can be regulated by the moisture of the neighboring air.

Gas sensing can also be achieved based on variation of non-electrical properties like optical, calorimetric, gas chromatograph, and acoustic sensing. Optical sensors rely on spectroscopy, which uses emission spectrometry and absorption. The principle of absorption spectrometry is based on absorption of the photons at specific gas wavelengths; the absorption depends on the concentration of photons. Infrared gas sensors operate on the principle of molecular absorption spectrometry; each gas has its own particular absorption properties to infrared radiation with different wavelengths. In general, optical sensors could attain better selectivity, sensitivity, and stability in comparison to non-optical methods.

Calorimetric sensors are solid-state devices. The sensitive elements consist of small ceramic “pellets” with varying resistance depending on the existence of target gases. They detect gases with a substantial variation of thermal conductivity with reference to the thermal conductivity of air (e.g., combustible gases). Gas chromatography is a classic analytical method with exceptional capabilities for separation as well as high selectivity and sensitivity. However, gas chromatograph miniaturization is challenging. Other sensors which can be incorporated in the present technology are illustrated below.

| | | Physiological/ |
|---|---|---|
| Sensor | Environmental | Biometric |
| GPS [lat/lon to supported precision (e.g. roughly 64 bits/sample)] | x | |
| 9-axis IMU [e.g. 16-bit samples x 9 (XYZ on three sensors). 10-100 Hz sample rate | x | X |
| when active.] | | |
| Humidity [e.g. 8-bit samples, 0.1 Hz sample rate.] | x | |
| Barometric Pressure [e.g. 24-bit samples, 50 Hz sample rate.] | x | X |
| Ambient Temperature [May be included in pressure sensor. E.g. 8-bit samples | x | |
| (0.5° F. resolution). 0.1 Hz sample rate.] | | |
| Core body Temperature (e.g. non-contact infra-red body temperature sensor, or | | X |
| a thermometer or a thermography sensor) | | |
| MEMS Microphone [e.g. Single microphone because no significant directionality in | x | X |
| this form factor with multiple mics because of limited spacing. 16-bit sample rate, 48 | | |
| kHz sample rate, inline compression.] | | |
| Ambient Light [e.g. Single band (eye spectral response) and 8-bit samples (log | x | |
| scale), 0.1 Hz sample rate.] | | |
| Ionization radiation detector [CsI: TI scintillator, personal dosimeter, 8-bit samples | x | |
| (log scale), e.g. 0.1 Hz sample rate.] | | |
| Radiofrequency and Terahertz submillimeter radiation sensor stack ( e.g. field | | X |
| strength sensor in the range of about 1 kHz to about 50 GHz or Passive RF | | |
| energy burst (or modulated) in the 10 MHz-6 GHz range) | | |
| Pulse rate, respiratory rate, body temperature, blood pressure, and peripheral | | X |
| capillary oxygen saturation [4 × 4 red, green, and blue (RGB) LED array 12-bit | | |
| samples, 100 Hz sample rate.] | | |
| Electric potential sensor [ECG and AC bioimpedance, skin conductance, galvanic | | X |
| skin response (GSR), electrodermal response (EDR), electrodermal activity (EDA), | | |
| 12-bit samples, 16 kHz sample rate] | | |
| Ballistocardiography vibroacoustic and physiological and environmental infrasound | x | X |
| sensor [far infrasound-to-far ultrasound physiologic sensor, 16-bit samples, 48 KHz | | |
| sample rate, pre-compression . . .] | | |

Other sensors include vO2, vCO2, respiratory quotient (RQ), and energy expenditure (EE), B-2 & B-52 sorties: ketones and hydration, CO2 excretion, tidal capnography.
SpO2, SaO2, PaO2 sensor suite.
Integrated Biopotential and Bioimpedance AFE.
Low-Power, Integrated In-Ear Heart-Rate Monitor.
Finger Heart Rate and Pulse Oximeter Smart Sensor.
Thermistor on flex foil (temperature sensor).
Non-contact infrared temperature sensor.
Optical Pulse Oximeter and Heart-Rate Sensor.
Health Sensor (hSensor) platform supporting the measurement of motion, precision skin temperature, bio-potential measurements (including ECG, EMG, EEG, and DC-potential EEG) and reflective PPG (including pulse oximetry and heart rate).
GSR.
Ballistocardiography (BCG) and seismocardiography (SCG) sensors.
DC-potential electromagneticcardiogram (EMCG) sensor.
Circadian rhythm sensor.
Ultra-Wide-Band sensor breathing analyzer.
Electrochemical Sensor AFE.
Continuous and/or pulsed Infrasound-to-ultrasound.
Continuous and/or pulsed electromagnetic frequency (EM).
Continuous and/or pulsed radio frequency (RF).
Continuous and/or pulsed lo/hi energy laser.
Continuous and/or pulsed magnetic resonance.

The standard electrocardiogram (ECG), measures the electrical activity of heart and is widely used in clinical settings, is difficult to integrate into wrist worn devices. Instead, most current solutions focus on photoplethysmography (PPG), which operates by shining light onto the body and measuring the amount of reflection which is modulated by the blood flow. This means that flow, volume, pressure, etc., factors and morphological components of the waveform (such as P-waves and T-waves in the ECG and heart rate variability cannot be directly reported and are indirectly inferred using biophysiology-independent black-box AI algorithms. Moreover, as PPG sensing uses a light source (typically an LED), it inherently consumes a large amount of power.

Integration of ECG into a wearable device such as standard watch at the wrist is challenging due to the need for electrodes to be placed on either side of the heart. If electrodes are placed on just one side of the heart, the collected signals reduce in amplitude and become increasingly small the further away from the heart. Placing electrodes on just one arm, the time domain ECG signal reaches a 0 dB Signal-to-Noise Ratio at (approximately) the elbow. It is possible, however, to place one electrode on one wrist, and then touch a second electrode with the other hand as done with the Apple™ watch. As the two sensing connection points are on either side of the heart a high Signal-to-Noise Ratio (SNR) ECG can be collected.

Sensors to be included in the wearable device may include any other sensor that can monitor vibroacoustic and electrical activity generated by the body and/or brain of a user without or without making contact with the body.

Foldable sensor

In certain embodiments, the sensor device 12 of FIG. 1 comprises a plurality of sensors supported by two or more substrates and which can have a folded configuration or an unfolded configuration. In the folded configuration, the sensor device 12 has multiple substrate layers supporting the plurality of sensors. The multiple layers of the sensor device 12 may be formed through a folding mechanism.

The sensor device includes a substrate, a join member, a substrate, a join member, and a substrate. The join members permit the substrates to be folded relative to each other into the folded configuration. Sensors and/or other electronic components may be positioned on one or more of the substrates. The substrates may include printed circuit boards (PCBs) and the sensors may be attached to the PCBs. It will be appreciated that for a given substrate, the sensors may be positioned on one or both sides of the substrates.

In use, the foldable sensor device can be placed against, or close to, a body part of a user, such as the user's skin, when in the folded configuration with one side of the substrates facing the body part.

All or a portion of the join members may be flexible and/or formed of a flexible material. Alternatively, or additionally, the join members may form a hinge, accordion-like structure, and/or any other suitable structure for allowing the substrates to be positioned in a multi-layer configuration. The join members may be thinned or otherwise dimensioned to enable the foldable sensor device to be folded.

One or more of the substrates may be made of the same material as the join members and include a stiffener material for strengthening and rendering less flexible. In other embodiments, one or more of the substrates may be made of a different material than the join members.

In certain embodiments, the substrates and/or join members may be manufactured as a single piece. In other embodiments, the substrates and the join members may comprise different components which are attached to each other.

In an unfolded configuration of the foldable sensor device, the substrates and join members are co-planar. In the folded configuration, the substrates may be stacked one above each other.

The arrangement of the sensors on the substrates may be such that in use, when the foldable sensor device is positioned on or near the body of the subject, some of the sensors of the foldable sensor device may face the user's body and/or some of the sensors of the foldable sensor device may face outwardly towards the environment, away from the user's body. The sensors facing towards the user's body may capture physiological data of the user. The sensors facing away from the user's body may capture environmental data describing the environment surrounding the user. Both types of data, physiological and environmental, may be captured simultaneously by the foldable sensor device. The data capture may be continuous or intermittent.

In other embodiments (not shown), at least a portion of at least one sensor may be formed within the body of the substrates. Such sensor or sensor portion may include filtering elements, such as a copper plate, light filter, and/or layer of piezoelectric material that reacts to being bent. The layer of piezoelectric material may function as a vibroacoustic sensor.

In certain embodiments, the sensors facing the user's body include a vibroacoustic sensor, a PPG/SpO2 sensor, and an electric potential sensor. The sensors facing away from the user's body include a pressure sensor, a temperature sensor, a humidity sensor, a light sensor, and an inertial measurement unit (IMU). In other embodiments, any other combination of sensors for detecting physiological and/or environmental signals may be used in the foldable sensor device. The types of sensors that can be used with the present technology is not particularly limited, and certain example sensors are described herein.

The substrates and join members may include other electronic components, such as communication components including an antennae, power sources including a battery, storage devices including flash memory which may be removable, processors, a Universal Serial Bus (USB) port or other data transmission port, shielding components, grounding components and/or a signal amplifying component. One or more batteries may be included in the foldable sensor device. The batteries may be attached to the substrates. When the foldable sensor device 200 is folded, the batteries may be sandwiched between the substrates. The batteries provide power to the sensors and/or other electronic components of the foldable sensor device. The antenna may be incorporated in the join members.

The foldable sensor device may include a storage unit for storing data collected by the sensors. The storage unit may be communicatively coupled to the sensors to receive the data captured by the sensors. The storage unit may be accessed by a processor of the foldable sensor device. The data stored on the storage unit may be accessed via the USB port of the foldable sensor device and/or via a wireless communication protocol, such as Wi-Fi or Bluetooth. The storage device may be removable, such as a removable flash memory device.

The foldable sensor device may have various shapes. For example the foldable sensor device may be derived from a polyhedron which is flattened (unfolded configuration) then folded (folded configuration). In one example, the polyhedron may have six faces which when flattened and then folded would create six substrate layers. The arrangement of the sensors on the faces of the substrates may differ from that as illustrated.

### Wearable Devices

### Watch Configuration

In certain embodiments, the wearable device has a watch configuration.

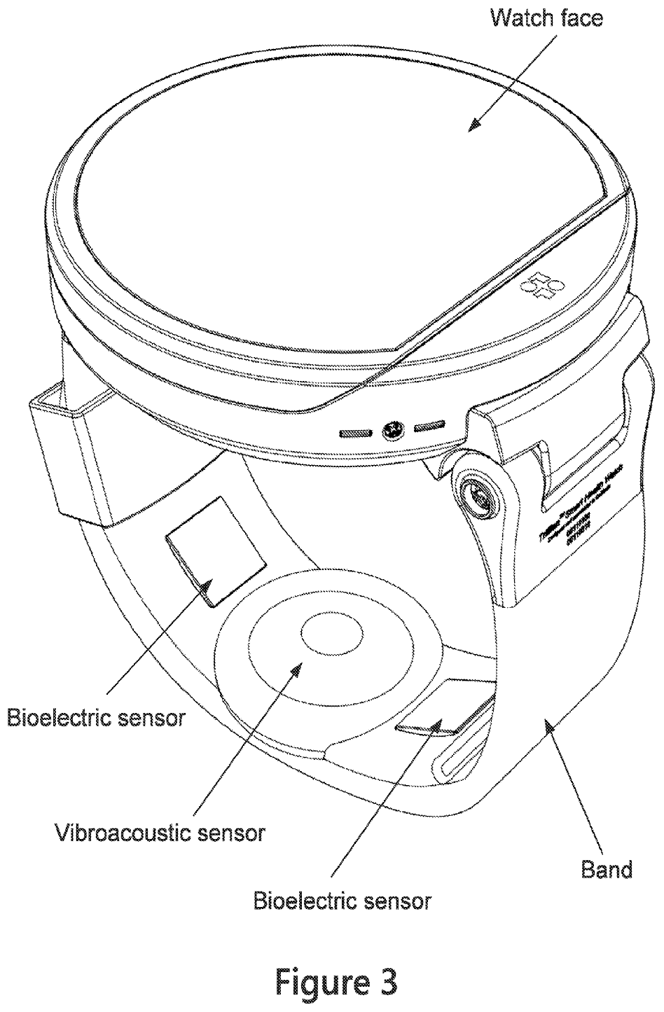

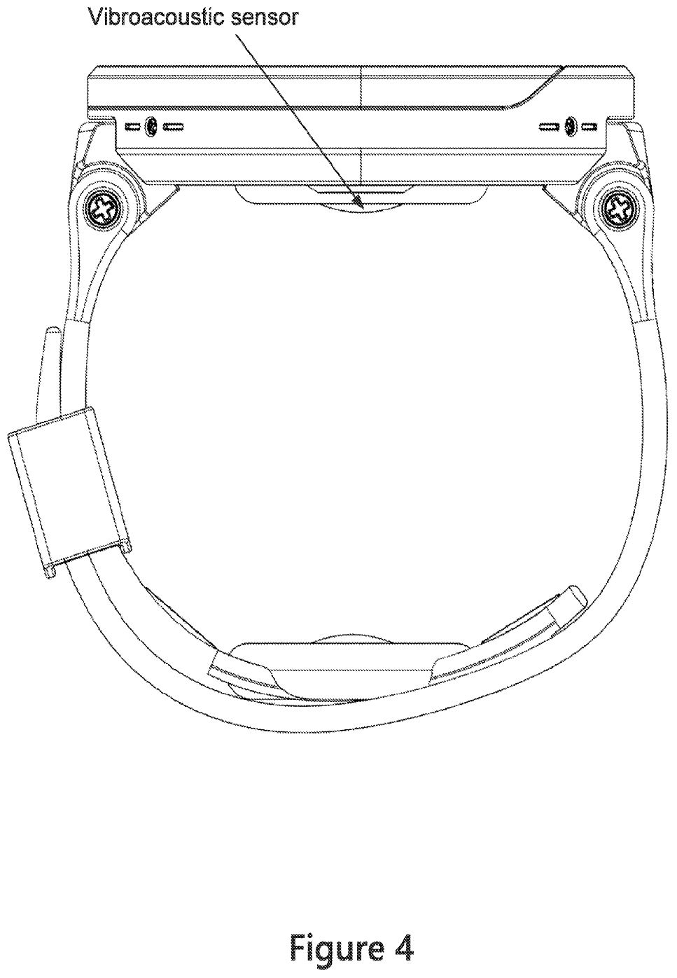

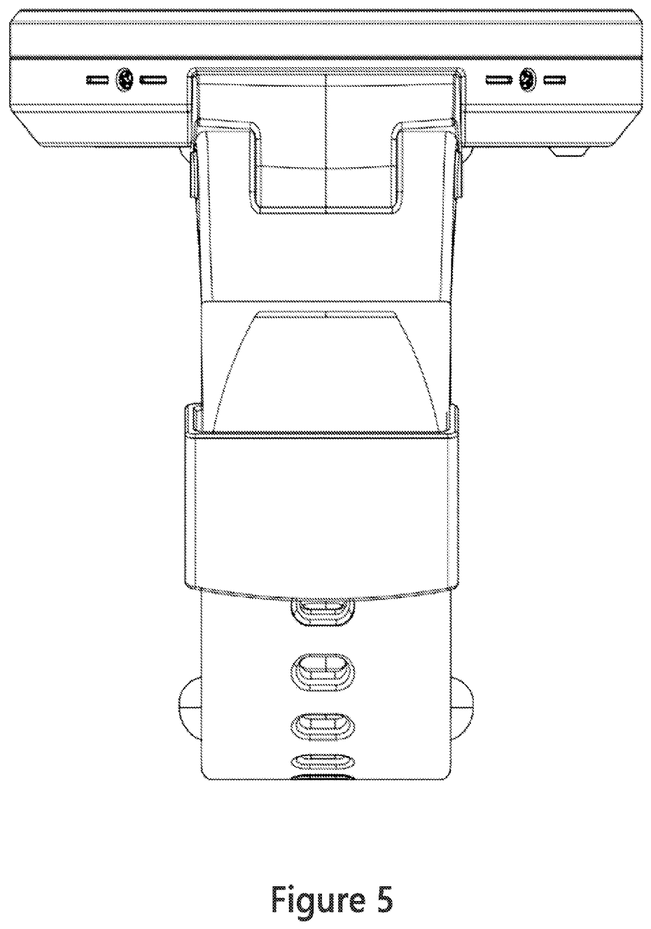

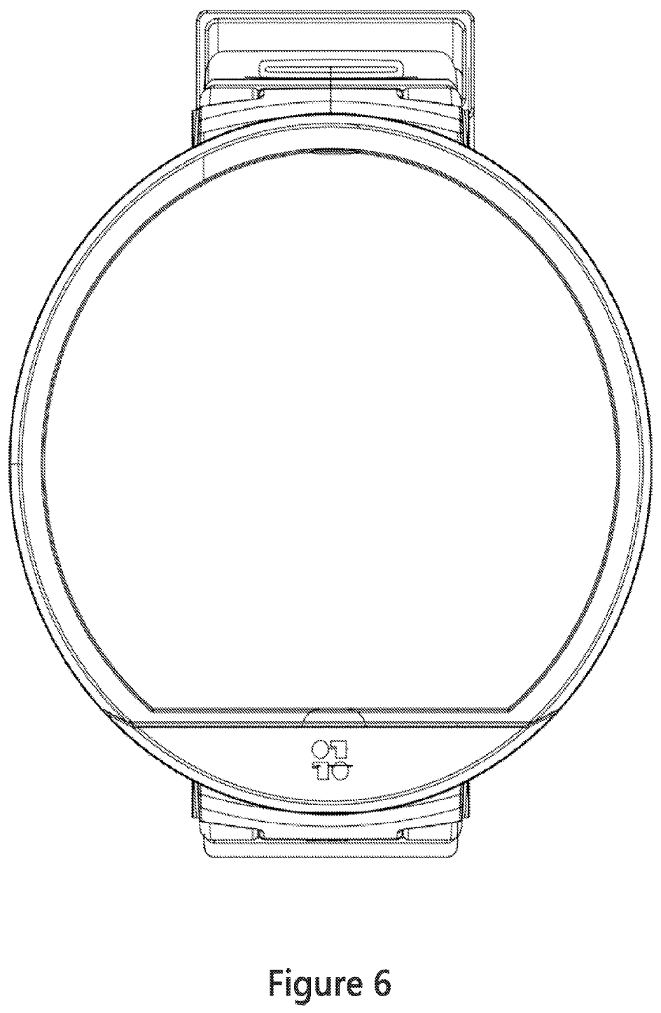

One embodiment of the wearable device with a watch configuration is illustrated in FIGS. 3-6. FIG. 3 is a perspective view of the wearable device which is configured as a watch. FIG. 4 is a side view of the watch of FIG. 3. FIG. 5 is an end view of the watch of FIG. 3. FIG. 6 is a top view of the watch of FIG. 3.

The wearable device comprises a band and a watch face (display). The wearable device includes a first vibroacoustic sensor positioned on an inner side of the band. The first vibroacoustic sensor is wrist-facing when the watch is worn by the user on the wrist of the user. A second vibroacoustic device is provided on a back side of the watch face. Two bioelectric sensors are provided on the inner side of band, one on either side of the vibroacoustic sensor.

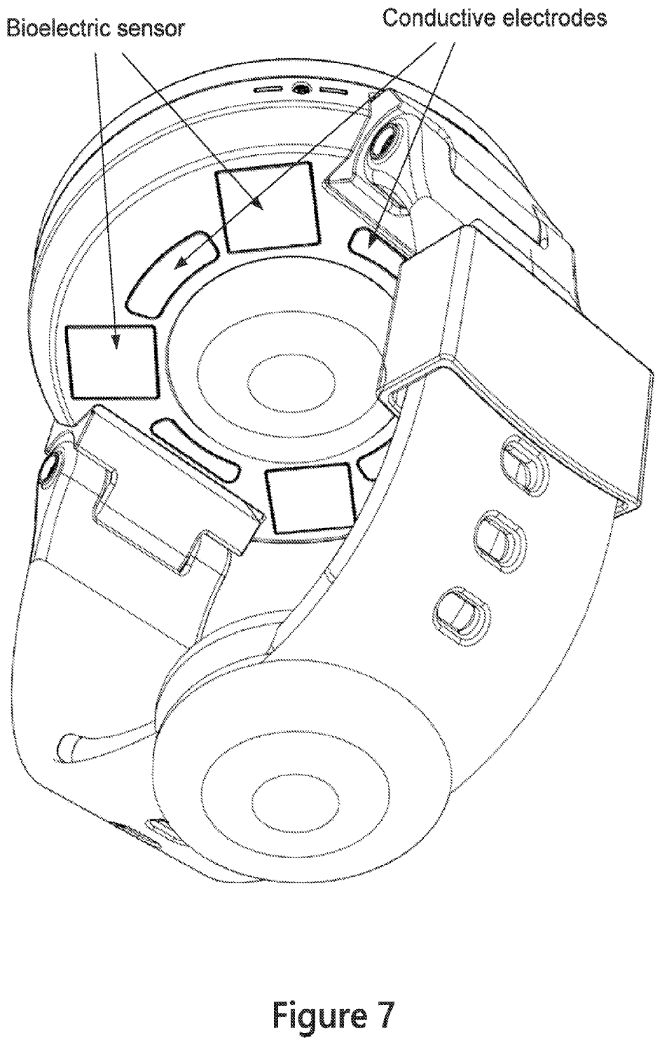

Another embodiment of the wearable device with a watch configuration is illustrated in FIG. 7. The embodiment of FIG. 7 differs from that of FIGS. 3-6 in that a third vibroacoustic sensor is provided on the band. The third vibroacoustic sensor is positioned on the outer side of the band. The third vibroacoustic sensor faces away from the wrist when the watch is worn by the user on the wrist of the user. This can allow placement of the third vibroacoustic sensor on other body locations of the user for vibroacoustic sensing.

In yet other embodiments (not shown), the wearable device may include a single vibroacoustic sensor on the outer side of the band.

In yet other embodiments (not shown), the wearable device may include a vibroacoustic sensor on the outer side of the band and a vibroacoustic sensor on the inner side of the band.

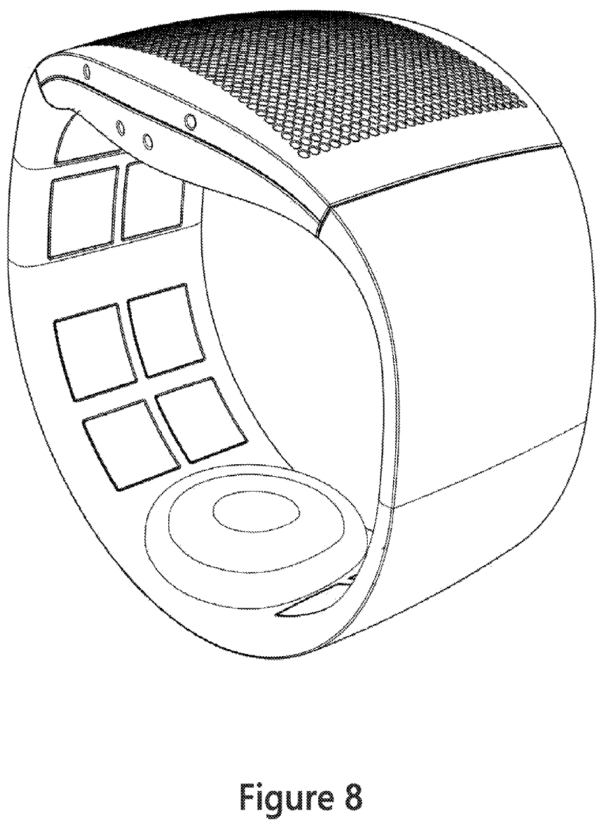

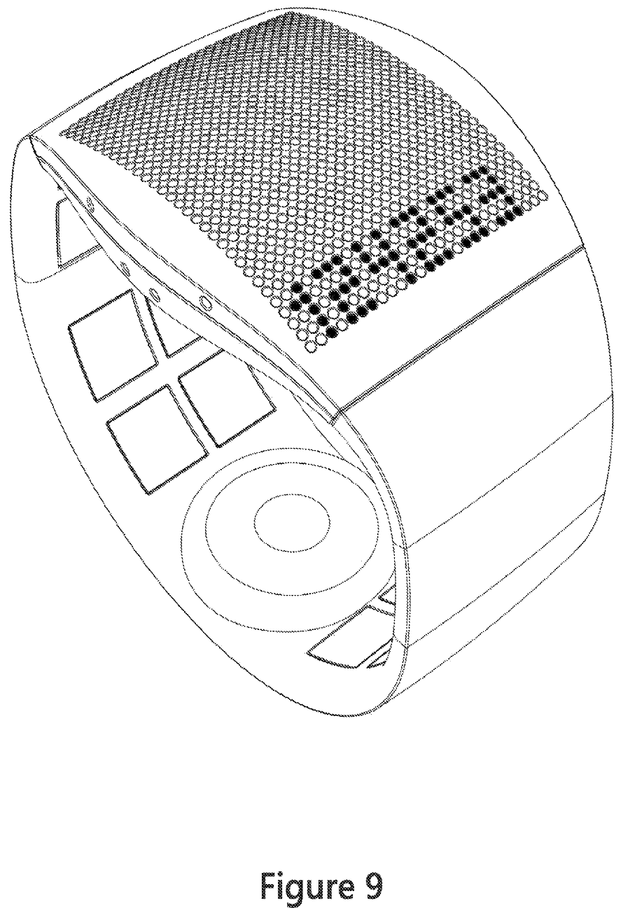

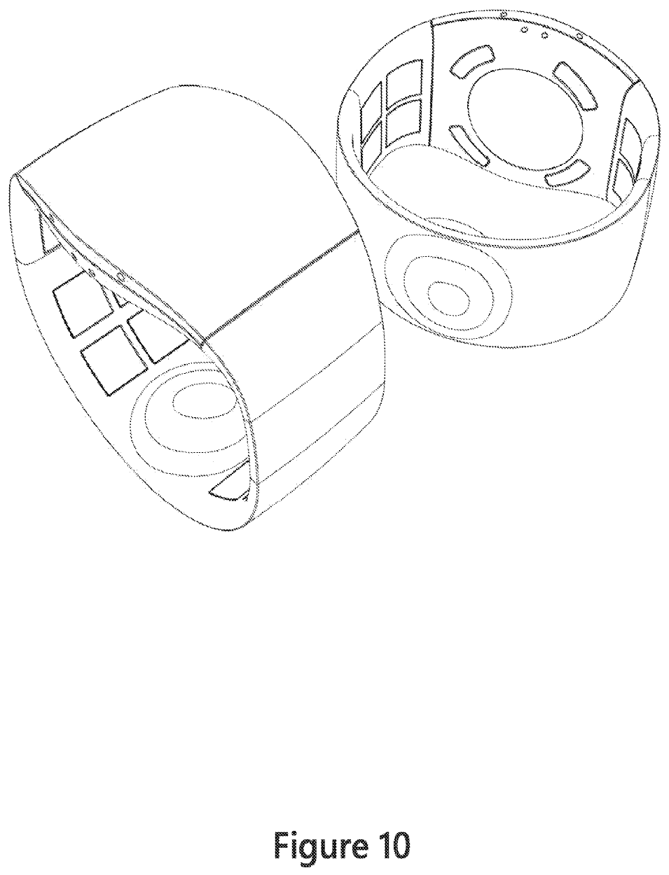

FIGS. 8-10 illustrate other embodiments of the wearable device which can be worn around the wrist or ankle of the user.

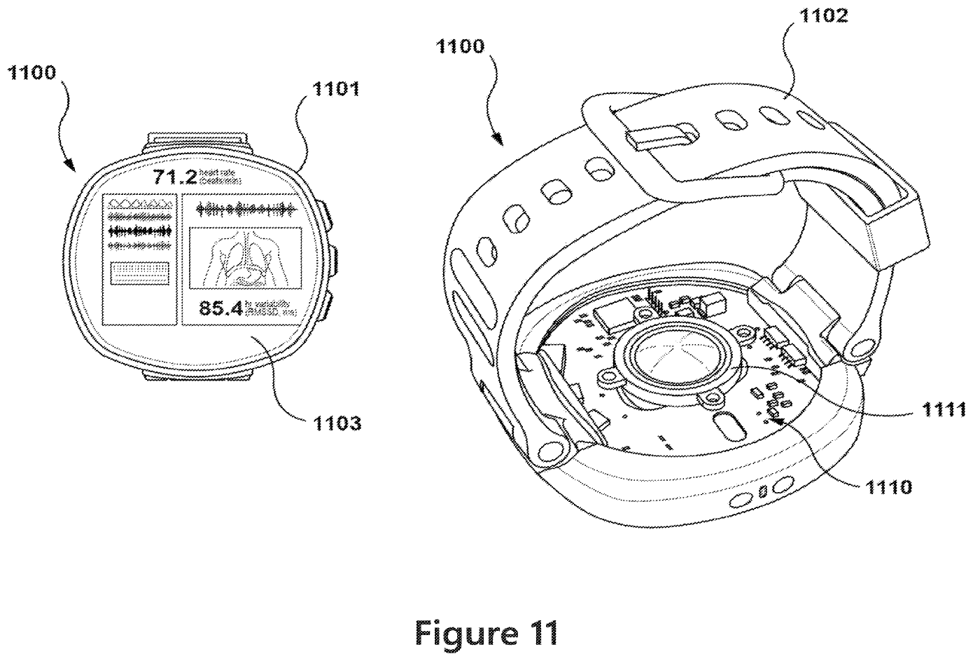

FIG. 11 illustrates yet another embodiment of the wearable device which can be worn around the wrist of the user.

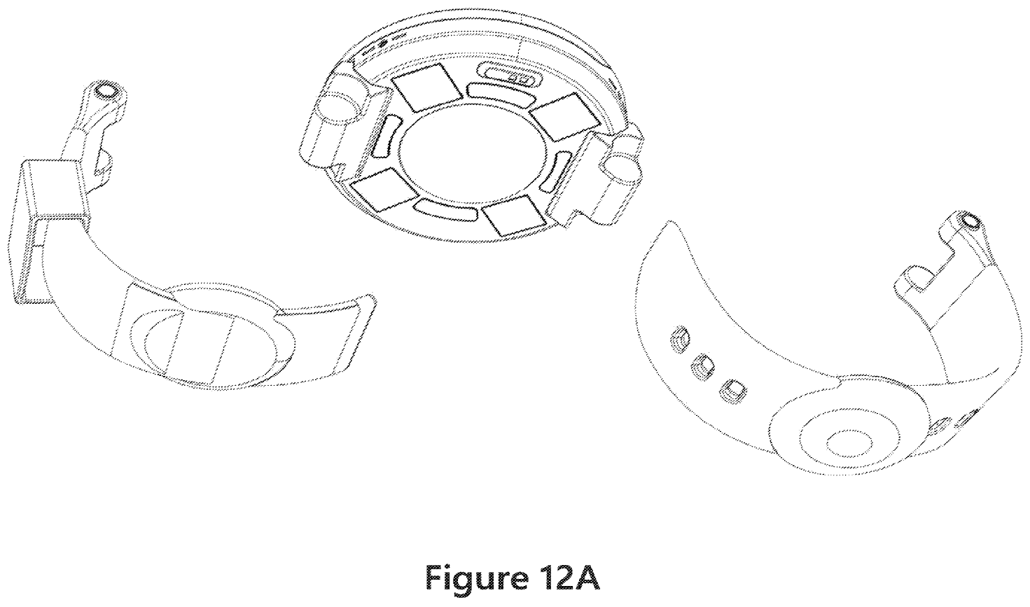

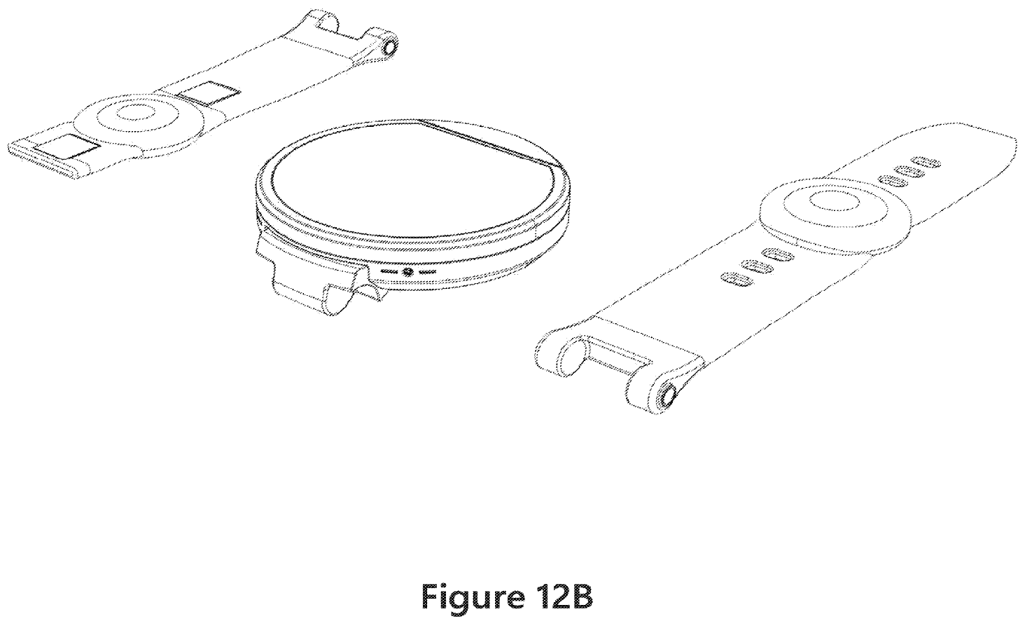

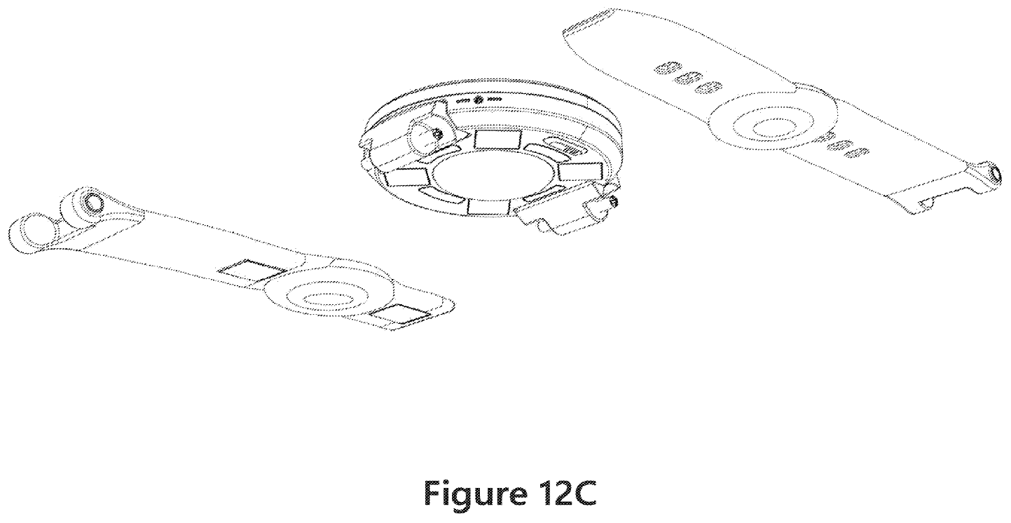

FIGS. 12A-C illustrate a further embodiment of the wearable device in which the band and the display are modular.

The modular wearable device may comprise a display unit, a left wrist band unit, and a right wrist band unit. Each of the display unit, the left wrist band unit, and the right wrist band unit may be modular and function as independent vibroacoustic sensors. In certain embodiments, each of the display unit, the left wrist band unit, and the right wrist band unit may be powered by its own power supply (i.e. battery) and a wireless transmission unit to allow for telemetry and communication of sensor activation instructions and acquired sensor data therebetween. Therefore, each unit may be able to independently function even when detached. Wireless transmission may additionally include power transmission with each unit capable of charging the battery within each unit when placed on or near a wireless charger. As there are now many universal standards for wireless inductive charging such as Qi-Certification, and an ongoing transition to almost all new smartphones, smartwatches, being cordless charging compatible, keeping all units charged will not be an issue.

The units of the modular wearable device may be detachably attachable together. Each unit may comprise one or more sensors that can be used together or separately as satellite units to detect and collect accurate data from the body. Supporting sensors on different modular components of such a device can provide a more efficient self-directed monitoring of both a subject which is wearing the device, accessing parts of the body far from the wrist of clinical interest as well as collecting environmental determinants of health data around the subject for context. Given sensors can thereby be positioned closer to the object that they are monitoring for more efficient signal pick-up. This can be advantageous for further processing of the signals detected in terms of signal-to-noise ratios.

In a connected state, the wristband may be in an open state and two or more vibroacoustic sensors may be used by partially wrapping the wristband around a desired evaluation location temporarily. The desired evaluation location may be a body extremity such as a wrist, arm, ankle, or calf from which heart rate, blood pressure, temperature, and extremity blood flow may be monitored. Alternatively, the desired evaluation location may be to target a specific organ such as lungs for chronic pulmonary disease and diffuse alveolar damage evaluation, a liver for hepatic function and inflammation evaluation, or kidney for glomerular filtration rate and abnormalities such as kidney stones or cysts.

The display may display data collected by the at least two sensors, such as physiological data corresponding to the user and/or environmental data corresponding to the environment surrounding the user. The wearable device may store and/or transmit the collected sensor data. The wearable device may include a wireless networking device to transmit the data via a wireless interface, such as via Wi-Fi and/or Bluetooth.

The wearable device may be configured to have a number of operating modes including one or more of: a continuous data collection mode; an intermittent data collection mode at fixed time-intervals; and a threshold-triggered data collection mode. A given sensor in the wearable device may have a different collection mode than another given sensor. For example, the GPS and pressure sensors may operate under the continuous data collection mode whereas the vibroacoustic sensor may operate under the threshold-triggered data collection mode.

### Other Configurations

In other embodiments, the wearable may have any suitable form factor, such as, without limitation: a strap, a band aid, a patch (e.g. drug delivery patch with or without medication), a watch, a bandage, an item of jewelry, a head piece (e.g. helmet such as a military, safety/protective or sports helmet, hat, headband), an eye piece (e.g. goggles, spectacles, monocles, safety spectacles, sun glasses, visors (e.g. for sports, safety, military) or other eye wear), an ear piece (e.g. hearing aid, earphones, earbuds, headphones, ear covering shields), a mouth piece (e.g. mouthguards, self-contained breathing apparatus (SCBA)), a device for the face (e.g. masks or visors for surgical, sport, or other protective use; ski masks; swimming goggles), devices for the wrist or arm (e.g. smart watches, watches, connected bracelets, wearable activity trackers), a collar, an item of clothing (e.g. for protection against accidents and against fire; body armor; footwear such as boots, shoes, trainers; vests, hijabs, niqabs, abayas, mittens, gloves; uniforms such as for sport or military, belts), a support (e.g. knee, wrist, elbow, hip and shoulder pads for athletic or medical use; shin guards, bedding), devices for invasive use (e.g. a cervical collar), collars for animals, jewelry, blankets, pillows, bedding or cushions.

### Methods

### Methods for Collecting Data

In certain embodiments, there are provided methods for collecting data. In one or more aspects, the method or one or more steps thereof may be performed by a computing system, such as the computing environment 100. All or a portion of the steps may be executed by the wearable device described herein, such as by a processor and memory of the wearable device. Alternatively, all or a portion of the steps may be executed by a processor external to the wearable device, such as a server.

The method or one or more steps thereof may be embodied in computer-executable instructions that are stored in a computer-readable medium, such as a non-transitory mass storage device, loaded into memory and executed by a CPU. Some steps or portions of steps in the method may be omitted, changed in order, and/or executed in parallel.

In certain aspects, a method for collecting data comprises: the processor causing an instruction to be provided to the user on placement of the wearable device on a first body position of the user for receiving a first set of user vibroacoustic sensor data; the processor receiving a first set of user vibroacoustic sensor data from the wearable device responsive to the positioning of the wearable device; the processor determining that the first set of user vibroacoustic sensor data satisfies a first set of sensor acquisition criteria; the processor causing an instruction to be provided to the user on placement of the wearable device on a second body position of the user for receiving a second set of user vibroacoustic sensor data; the processor receiving the second set of user vibroacoustic sensor data from the wearable device responsive to the positioning of the wearable device; and the processor determining that the second set of user vibroacoustic sensor data satisfies a second set of sensor acquisition criteria.

The instruction may comprise a command provided from the wearable device such as one or more of an audio signal, a visual signal, a haptic signal, etc. For example, when the wearable device has a watch configuration with a watch face, a picture depicting a location on the body on which the wearable device should be positioned for the data capture may be displayed on the watch face.

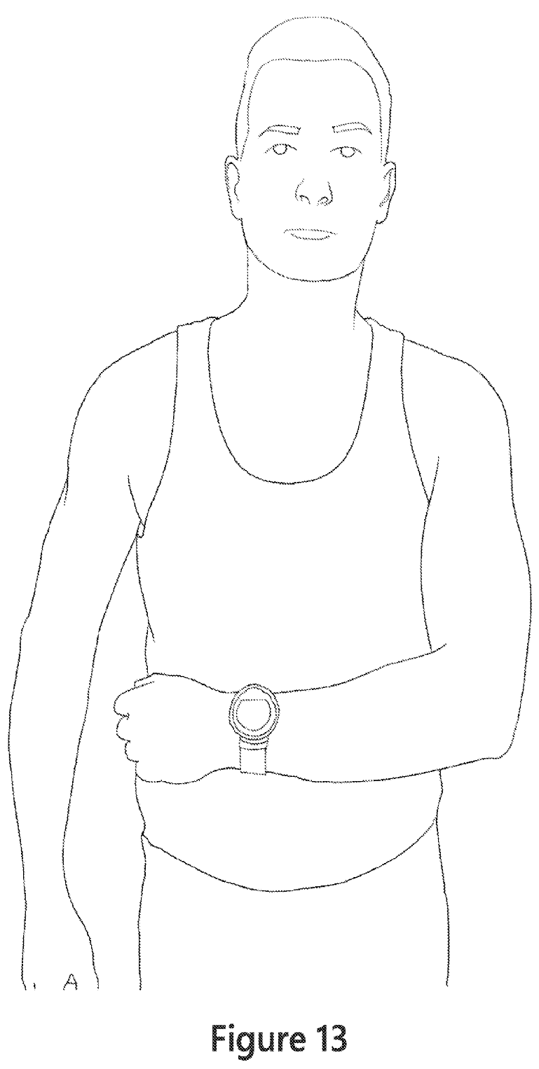

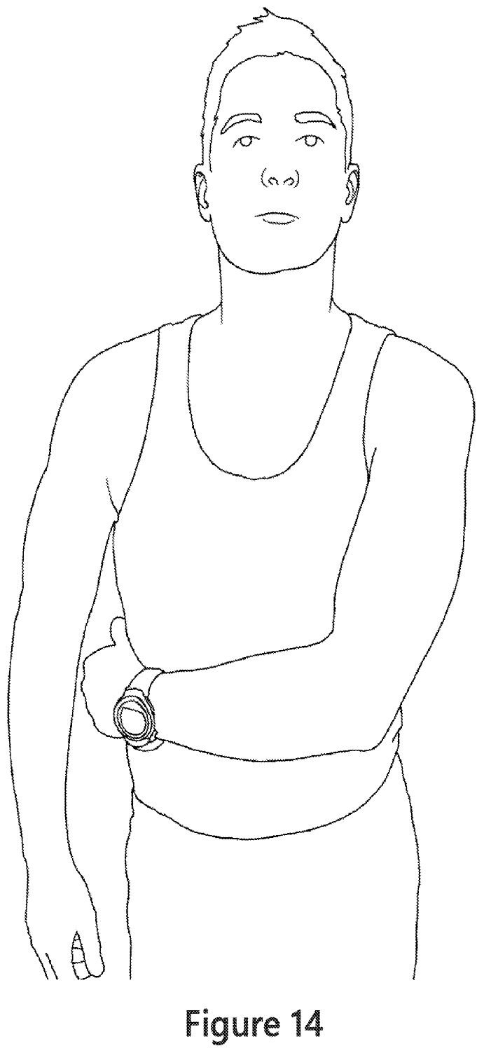

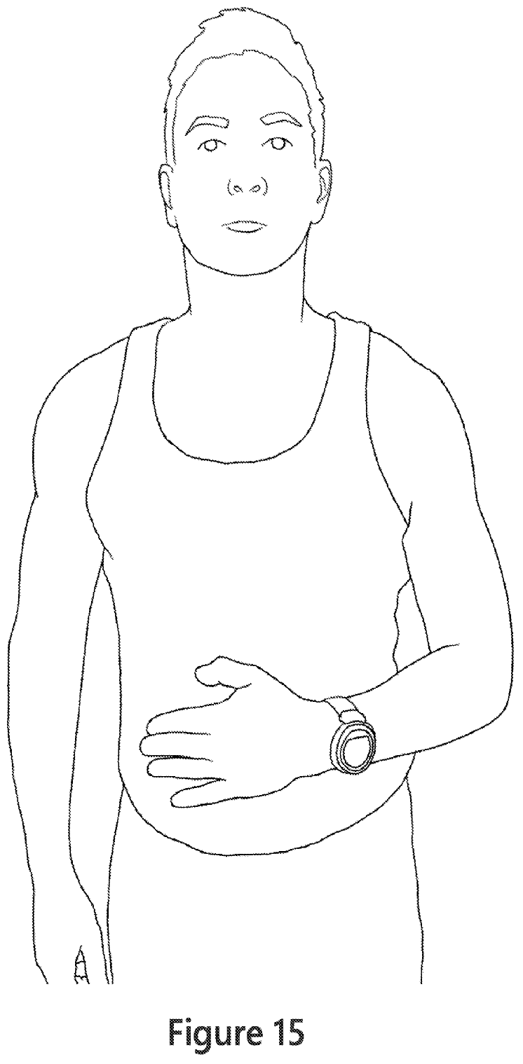

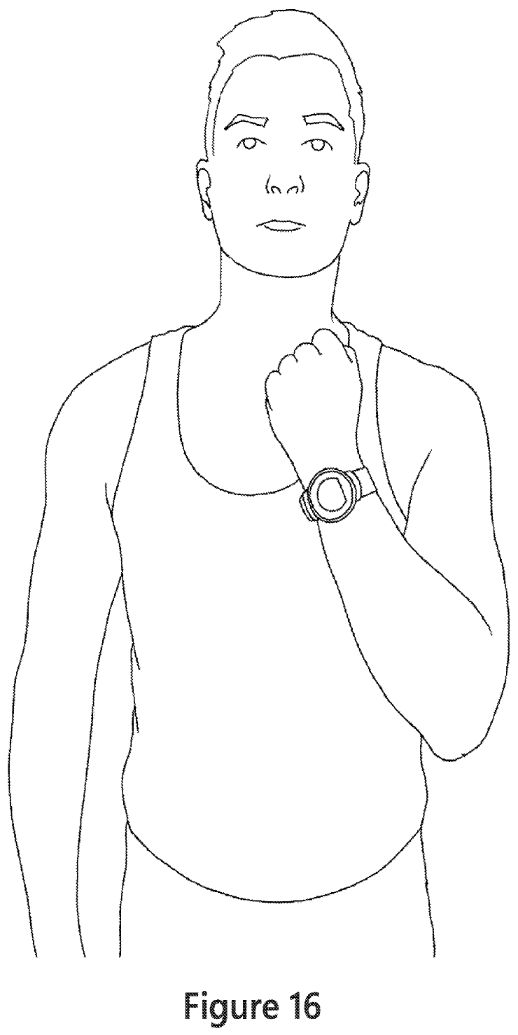

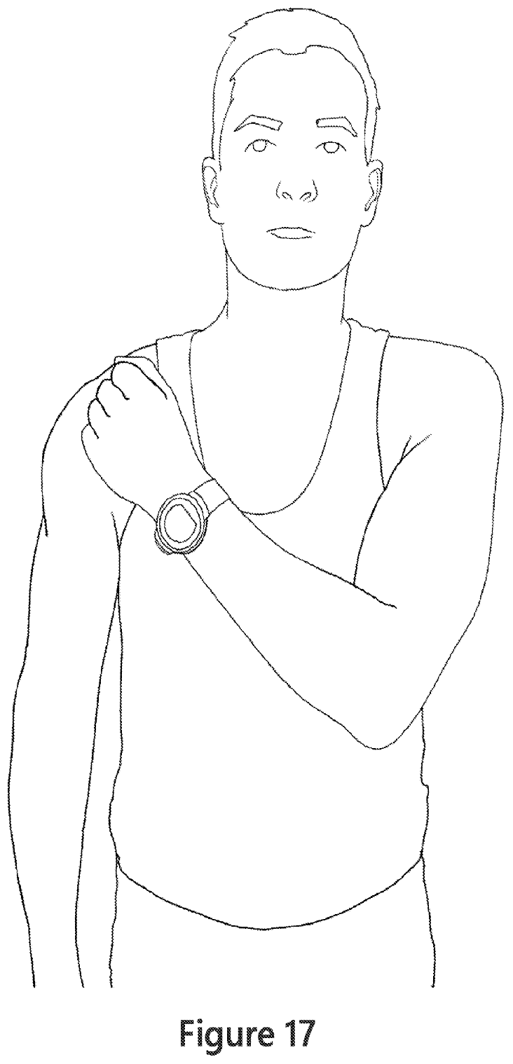

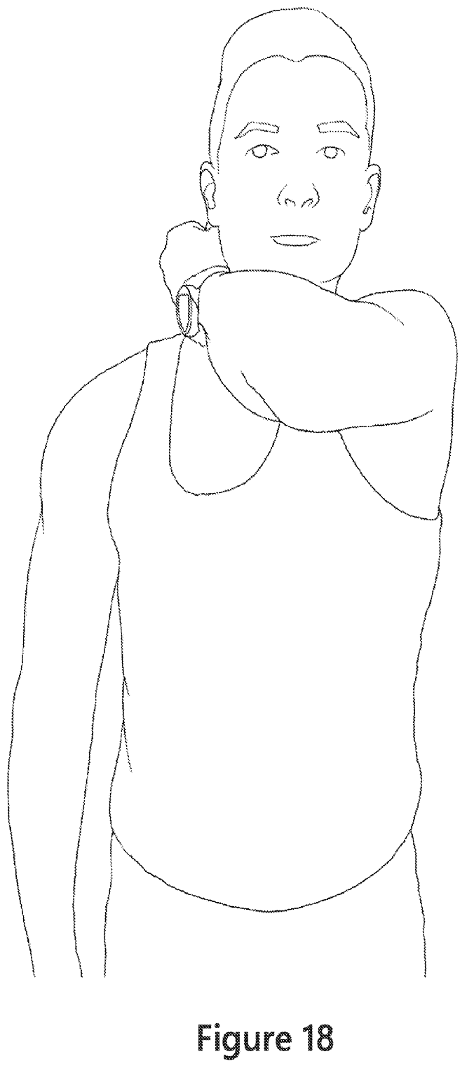

Example locations are shown in FIGS. 13-18 in which the wearable device is positioned on: the gut (FIG. 13), renally (FIG. 14), gut (FIG. 15), lung—first auscultation point (FIG. 16), lung—second auscultation point (FIG. 17), carotid artery (FIG. 18).

The instruction may include a command regarding the order of the auscultation points and/or an identity of which auscultation point to target.

### Methods for Predicting or Monitoring a Condition of a User

In certain embodiments, there are provided methods for predicting or monitoring a condition of a user. In one or more aspects, the method or one or more steps thereof may be performed by a computing system, such as the computing environment 100. All or a portion of the steps may be executed by the wearable device described herein, such as by a processor and memory of the wearable device. Alternatively, all or a portion of the steps may be executed by a processor external to the wearable device, such as a server.

The method or one or more steps thereof may be embodied in computer-executable instructions that are stored in a computer-readable medium, such as a non-transitory mass storage device, loaded into memory and executed by a CPU. Some steps or portions of steps in the method may be omitted, changed in order, and/or executed in parallel.

The method may comprise receiving data from the at least two sensors of the wearable device. The data may comprise raw signal data captured by the sensors, or be a filtered and/or amplified data set.

The data may be input to a machine-learning algorithm (MLA). The MLA may have been trained to predict, based on data collected by the wearable device, whether an individual is subject to a condition, such as a medical condition. The condition may include a blood pressure of the user. The condition may comprise one or more of: chronic pulmonary disease, lung consolidation, diffuse alveolar damage, vascular injury, and/or fibrosis, pulmonary embolism, and disseminated intravascular coagulation (DIC) cardiac structural disorders such as left ventricular hypertrophy, carotid disease, or coronary disease.

The method may comprise outputting the predicted condition of the user and/or any other predictions that the MLA has made based on the data. A user interface describing the predictions may be displayed to the user or to a third party. In this respect, the processor may cause an alert to be sent to an electronic device such as a smart phone of the third party.

By condition is meant one or more of a medical condition, a cognitive load state, an alertness state and an activity of the user or a state of mind of the user.

### CLAUSES

1. A wearable device comprising:
   a vibroacoustic sensor directionally facing away from the wrist that can be placed against a body part of
   a user by movement and articulation of the arm and wrist.
2. The wearable device of clause 1, wherein the device comprises one or more of: a strap, a band aid, a patch, a watch, a bandage, an item of jewelry, or an item of clothing.
3. A wearable device comprising two or more vibroacoustic sensors which are positioned such that they can collect data from different auscultation points from a body of a user, when the wearable device is worn by the user.
4. The wearable device of clause 3, wherein the different auscultation points comprise a radial artery and an ulnar artery on a wrist region of the user.
5. The wearable device of clause 3, wherein the different auscultation points are located at two or more of: a heart region, a gut region, a renal region, a femoral artery region, and a carotid artery region.
6. The wearable device of any of clauses 3-5, wherein the vibroacoustic sensors are positioned such that they have at least a 10 degree separation for simultaneous vibroacoustic data acquisition from the different auscultation points.
7. The wearable device of any of clauses 3-6, wherein the vibroacoustic sensors are positioned on a band of the wearable device.
8. The wearable device of clause 7, wherein one vibroacoustic sensor is positioned on an outer face of the band and faces outwardly when the wearable device is worn by the user and another vibroacoustic device is positioned on an inner face of the band and faces inwardly when the wearable device is worn by the user.
9. The wearable device of clause 7 or clause 8 wherein the band includes a partially removeable band portion.
10. A wearable device configured to be worn on a wrist or an ankle of a user, the wearable device comprising:
   a first vibroacoustic sensor configured to contact the wrist of the user when the user wears the wearable device, and
   a second vibroacoustic sensor configured to contact a separate portion of the body onto which the user locates the wearable device.
11. A wearable device configured to be worn on a wrist of a user, the wearable device comprising:
   a first vibroacoustic sensor configured to contact a first position on the wrist of the user when the user wears the wearable device, and
   a second vibroacoustic sensor configured to contact a second position on the wrist of the user when the user wears the wearable device.
12. The wearable device of clause 11, wherein the first vibroacoustic sensor and the second vibroacoustic sensor are positionally distanced by at least 10 degrees to measure one or more arteries within the wrist from a different position.
13. The wearable device of clause 11 or clause 12, wherein the first vibroacoustic sensor and second vibroacoustic sensor are positionally distanced by up to 180 degrees to measure one or more arteries within the wrist from a different position.
14. The wearable device of any of clauses 11-13, wherein the first vibroacoustic sensor and the second vibroacoustic sensor each have a different bandwidth, thereby providing an overlapped combination to cover a wider bandwidth with optimal performance.
15. A method for collecting data comprising:
   causing an instruction to be provided to the user on placement of a wearable device on a first body position of the user for receiving a first set of user vibroacoustic sensor data;
   receiving a first set of user vibroacoustic sensor data from the wearable device responsive to the positioning of the wearable device;
   determining that the first set of user vibroacoustic sensor data satisfies a first set of sensor acquisition criteria;
   causing an instruction to be provided to the user on placement of the wearable device on a second body position of the user for receiving a second set of user vibroacoustic sensor data;
   receiving the second set of user vibroacoustic sensor data from the wearable device responsive to the positioning of the wearable device; and
   determining that the second set of user vibroacoustic sensor data satisfies a second set of sensor acquisition criteria.
16. A method of determining a condition of a user, the method comprising:
   receiving data related to the user simultaneously from two sensors measuring two different user body parts;
   inputting the data to a machine learning algorithm; and
   obtaining an output of whether the user has a condition.
17. The method of clause 16, wherein the sensor comprises a vibroacoustic sensor.
18. The method of clause 16 or clause 17, wherein the sensor comprises a bioelectric sensor.
19. The method of any of clauses 16-18, wherein the condition is a health screening, diagnosis, monitoring of disease progression, monitoring of reaction and effectiveness of a therapy.
20. A method for generating a unique identifier for a subject, the method executable by a processor of a computer system, the method comprising:
   obtaining biometric data relating to the subject from a vibroacoustic sensor and a secondary sensor simultaneously;
   extracting identification markers from the biometric data;
   generating the unique identifier from the extracted identification markers, wherein generating the unique identifier comprises identifying a given domain specific feature which has predetermined identity compared to other domain specific features.
21. A method of determining a physiological characteristic of a subject comprising the steps of:
   (a) positioning a vibroacoustic sensor in sensing proximity to a first location on the body of the subject;
   (b) positioning a second sensor in sensing proximity to a second location on the body of the subject;
   (c) converting the signals output from the sensors into one or more digital data streams;
   (d) processing the data stream with a machine learning algorithm to generate an indication of the physiological characteristic.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

It should be appreciated that the invention is not limited to the particular embodiments described and illustrated herein but includes all modifications

1. A wearable device comprising:
a vibroacoustic sensor directionally facing away from the wrist that can be placed against a body part of
a user by movement and articulation of the arm and wrist.

2. The wearable device of claim 1, wherein the device comprises one or more of: a strap, a band aid, a patch, a watch, a bandage, an item of jewelry, or an item of clothing.

3. A wearable device comprising two or more vibroacoustic sensors which are positioned such that they can collect data from different auscultation points from a body of a user, when the wearable device is worn by the user.

4. The wearable device of claim 3, wherein the different auscultation points comprise a radial artery and an ulnar artery on a wrist region of the user.

5. The wearable device of claim 3, wherein the different auscultation points are located at two or more of: a heart region, a gut region, a renal region, a femoral artery region, and a carotid artery region.

6. The wearable device of claim 3, wherein the vibroacoustic sensors are positioned such that they have at least a 10 degree separation for simultaneous vibroacoustic data acquisition from the different auscultation points.

7. The wearable device of claim 3, wherein the vibroacoustic sensors are positioned on a band of the wearable device.

8. The wearable device of claim 7, wherein one vibroacoustic sensor is positioned on an outer face of the band and faces outwardly when the wearable device is worn by the user and another vibroacoustic device is positioned on an inner face of the band and faces inwardly when the wearable device is worn by the user.

9. The wearable device of claim 7, wherein the band includes a partially removeable band portion.

10. A wearable device configured to be worn on a wrist or an ankle of a user, the wearable device comprising:
a first vibroacoustic sensor configured to contact the wrist of the user when the user wears the wearable device, and
a second vibroacoustic sensor configured to contact a separate portion of the body onto which the user locates the wearable device.

11. A wearable device configured to be worn on a wrist of a user, the wearable device comprising:
a first vibroacoustic sensor configured to contact a first position on the wrist of the user when the user wears the wearable device, and
a second vibroacoustic sensor configured to contact a second position on the wrist of the user when the user wears the wearable device.

12. The wearable device of claim 11, wherein the first vibroacoustic sensor and the second vibroacoustic sensor are positionally distanced by at least 10 degrees to measure one or more arteries within the wrist from a different position.

13. The wearable device of claim 11, wherein the first vibroacoustic sensor and second vibroacoustic sensor are positionally distanced by up to 180 degrees to measure one or more arteries within the wrist from a different position.

14. The wearable device of claim 11, wherein the first vibroacoustic sensor and the second vibroacoustic sensor each have a different bandwidth, thereby providing an overlapped combination to cover a wider bandwidth with optimal performance.

15. A method for collecting data comprising:
causing an instruction to be provided to the user on placement of a wearable device on a first body position of the user for receiving a first set of user vibroacoustic sensor data;
receiving a first set of user vibroacoustic sensor data from the wearable device responsive to the positioning of the wearable device;
determining that the first set of user vibroacoustic sensor data satisfies a first set of sensor acquisition criteria;
causing an instruction to be provided to the user on placement of the wearable device on a second body position of the user for receiving a second set of user vibroacoustic sensor data;
receiving the second set of user vibroacoustic sensor data from the wearable device responsive to the positioning of the wearable device; and
determining that the second set of user vibroacoustic sensor data satisfies a second set of sensor acquisition criteria.

16. A method of determining a condition of a user, the method comprising:
receiving data related to the user simultaneously from two sensors measuring two different user body parts;
inputting the data to a machine learning algorithm; and
obtaining an output of whether the user has a condition.

17. The method of claim 16, wherein the sensor comprises a vibroacoustic sensor.

18. The method of claim 16, wherein the sensor comprises a bioelectric sensor.

19. The method of claim 16, wherein the condition is a health screening, diagnosis, monitoring of disease progression, monitoring of reaction and effectiveness of a therapy.

20. A method for generating a unique identifier for a subject, the method executable by a processor of a computer system, the method comprising:
obtaining biometric data relating to the subject from a vibroacoustic sensor and a secondary sensor simultaneously;
extracting identification markers from the biometric data;
generating the unique identifier from the extracted identification markers, wherein generating the unique identifier comprises identifying a given domain specific feature which has predetermined identity compared to other domain specific features.

21. A method of determining a physiological characteristic of a subject comprising the steps of:
(a) positioning a vibroacoustic sensor in sensing proximity to a first location on the body of the subject;
(b) positioning a second sensor in sensing proximity to a second location on the body of the subject;
(c) converting the signals output from the sensors into one or more digital data streams;
(d) processing the data stream with a machine learning algorithm to generate an indication of the physiological characteristic.
